(19) United States
(12) Patent Application Publication
(10) Pub. No.: US 2003/0066102 A1
(43) Pub. Date: Apr. 3, 2003

(54) Plant gene for p-hydroxyphenylpyruvate dioxygenase

(76) Inventors: Carl Arthur Maxwell, Elkton, MD (US); Pablo Ariel Scolnik, Chapel, NC (US); Vernon Arie Wittenbach, Wilmington, DE (US); Steven Gutteridge, Wilmington, DE (US)

Correspondence Address: E I DU PONT DE NEMOURS AND COMPANY, LEGAL PATENT RECORDS CENTER, BARLEY MILL PLAZA 25/1128, 4417 LANCASTER PIKE, WILMINGTON, DE, 19805, US

(21) Appl. No.: 10/058,931

(22) Filed: Jan. 28, 2002

Related U.S. Application Data
(63) Continuation of application No. 08/882,969, filed on Jun. 26, 1997, now abandoned.
(60) Provisional application No. 60/021,364, filed on Jun. 27, 1996.

(51) Int. Cl. A01H 5/00; C12N 5/04; C07H 21/04; C12N 9/02; C12N 1/21

(52) U.S. Cl. 800/278; 800/320; 435/189; 435/417; 536/23.2; 435/252.33

(57) ABSTRACT

The invention relates to the isolation and modification of nucleic acid sequences encoding p-hydroxyphenylpyruvate dioxygenase enzyme from plants. These nucleic acid sequences were used to establish methods of identification of new herbicidal compounds that inhibit the activity of this enzyme, and to prepare new crop plants that are tolerant to the herbicidal action of inhibitors this enzyme. Chimeric genes comprising nucleic acid fragments containing all or part of the nucleic acid sequences encoding p-hydroxyphenylpyruvate dioxygenase may be used to produce active plant p-hydroxyphenylpyruvate dioxygenase enzyme in microorganisms, and to cause the production of modified forms of the enzyme in plants that may render such plants tolerant to inhibitors of the enzyme.

### FIELD OF THE INVENTION

This invention relates to the isolation and modification of nucleic acid encoding p-hydroxyphenylpyruvate dioxygenase enzyme from plants. These nucleic acid sequences were used to establish methods of identification of new herbicidal compounds that inhibit the activity of this enzyme, and to prepare new crop plants that are tolerant to the herbicidal action of inhibitors this enzyme. Chimeric genes comprising nucleic acid fragments containing all or part of the nucleic acid sequences encoding p-hydroxyphenylpyruvate dioxygenase may be used to produce active plant p-hydroxyphenylpyruvate dioxygenase enzyme in microorganisms, and to cause the production of modified forms of the enzyme in plants that may render such plants tolerant to inhibitors of the enzyme.

### BACKGROUND OF THE INVENTION

Bleaching herbicides affect plant chloroplasts by decreasing their chlorophyll and carotenoid content. Several bleaching herbicides are known to inhibit the enzyme phytoene desaturase, resulting in the accumulation of phytoene in treated plants. However, compounds of the benzoyl cyclohexane-1,3-dione type cause the accumulation of phytoene in plants but are not inhibitors of phytoene desaturase in vitro (Sandmann, G., et al. (1990) Pestic. Sci. 30:353-355). Subsequent work revealed that these compounds are effective inhibitors of p-hydroxyphenylpyruvate dioxygenase (p-hydroxyphenylpyruvate:oxygen oxidoreductase EC 1.13.11.27), a key enzyme in the biosynthesis of plastoquinones and tocopherols (Schulz, A., et al. (1993) FEBS Lett. 318:162-166). Based on the observation that phytoene desaturase requires a quinone as an electron acceptor, these authors postulated that by inhibiting p-hydroxyphenylpyruvate dioxygenase, these herbicides act indirectly on phytoene desaturase by blocking the biosynthesis of quinones.

The proposal that p-hydroxyphenylpyruvate dioxygenase is essential for carotenoid biosynthesis has received support from genetic studies in the plant model system Arabidopsis thaliana. Mutations in the pds1 and pds2 genetic loci result in mutant plants that accumulate phytoene. However, genetic mapping of these mutant genes indicates that they do not correspond to the gene encoding the enzyme phytoene desaturase. The pds1 mutation can be rescued by homogentisic acid, the substrate of p-hydroxyphenylpyruvate dioxygenase. Therefore, this mutation corresponds to a defect in the activity of p-hydroxyphenylpyruvate dioxygenase (Norris, S. R., et al. (1995) Plant Cell 7:2139-2149).

In light of these disclosures, p-hydroxyphenylpyruvate dioxygenase is a promising new target for new herbicidal compounds. Research aimed at discovering new herbicides based on this mode of action would be greatly facilitated by the isolation of the plant gene encoding this enzyme and by the functional expression of this gene in transgenic organisms. For example, active enzyme produced in recombinant microorganisms could be used to establish screening methods for the identification of novel active compounds and to obtain structural and mechanistic information useful to guide further chemical synthesis. Furthermore, isolation of this gene would facilitate research aimed at generating mutant, herbicide-tolerant versions of the enzyme that may confer herbicide resistance to transgenic plants.

A partial sequence of an Arabidopsis thaliana cDNA with homology to corresponding mammalian sequences encoding p-hydroxyphenylpyruvate dioxygenase has been identified (GenBank Accession No. T20952), but this truncated sequence is insufficient to identify an active plant p-hydroxyphenyl-pyruvate dioxygenase. WO 96/38567 A2 addresses the utility that would be attached to a DNA sequence of a p-hydroxyphenylpyruvate dioxygenase gene, but there is no biochemical evidence of function associated with the sequences disclosed.

### SUMMARY OF THE INVENTION

This invention pertains to the isolation and characterization of nucleic acid fragments encoding plant p-hydroxyphenylpyruvate dioxygenase enzymes. More specifically, this invention pertains to isolated nucleic acid fragments encoding the p-hydroxyphenylpyruvate dioxygenase enzymes from Arabidopsis thaliana and Zea mays.

This invention also pertains to the production of active plant p-hydroxy-phenylpyruvate dioxygenase enzyme in E. coli. In one embodiment, a chimeric gene comprising a nucleic acid fragment encoding a polypeptide that possesses p-hydroxyphenylpyruvate dioxygenase activity, operably linked to regulatory sequences that direct gene expression in E. coli, is claimed. In another embodiment, a plasmid vector comprising said chimeric gene is disclosed. In yet another embodiment, a transformed E. coli comprising a chimeric gene consisting of a nucleic acid fragment encoding a polypeptide that possesses p-hydroxy-phenylpyruvate dioxygenase activity is disclosed.

This invention also pertains to a method of identifying substances that inhibit the rate of the reaction of p-hydroxyphenylpyruvate dioxygenase enzyme. In one embodiment, the invention pertains to an assay for the detection of inhibitors of p-hydroxyphenylpyruvate dioxygenase wherein a polypeptide derived from a transformed E. coli that displays p-hydroxyphenylpyruvate dioxygenase activity is incubated in the presence of a test substance. Following incubation, p-hydroxyphenylpyruvate dioxygenase enzymatic activity is measured wherein a reduction of enzymatic activity is indicative of the inhibitory capacity of the test substance. Enzymatic activity can be measured by any appropriate means, including but not limited to oxygen utilization, carbon dioxide release, homogentisate production, and loss of p-hydroxyphenylpyruvate. Results are quantified by radiometric, colorimetric or chromatographic means.

In another embodiment, this invention pertains to plants that are substantially tolerant to the application of at least one compound that inhibits the rate of the reaction of p-hydroxyphenylpyruvate dioxygenase. Plants may be rendered tolerant by overexpression of the wild-type p-hydroxyphenylpyruvate dioxygenase, by expression of a naturally-occuring resistant variant of this enzyme, or by expression of an altered form of p-hydroxyphenylpyruvate dioxygenase that is resistant to the action of compounds that are inhibitory to the wild-type enzyme.

A further embodiment of the invention is an isolated nucleic acid fragment comprising a member selected from the group consisting of:

(a) an isolated nucleic acid fragment as set forth in SEQ ID NO: 16;

(b) an isolated nucleic acid fragment that is essentially similar to an isolated nucleic acid fragment as set forth in SEQ ID NO: 16; and

(c) an isolated nucleic acid fragment that is complementary to (a) or (b).

### BRIEF DESCRIPTION OF THE DRAWINGS AND SEQUENCE DESCRIPTIONS

The invention can be more fully understood from the following detailed description and the accompanying drawings and the sequence descriptions which form a part of this application.

FIG. 1 presents a partial nucleic acid sequence of an expressed sequence tag (EST) bearing GenBank Accession No. T92052 obtained from an Arabidopsis thaliana cDNA library. This sequence was contained in clone 91B13T7 of the library.

FIG. 2 presents the nucleic acid sequence of the cloned cDNA encoding a full-length form of Arabidopsis thaliana p-hydroxyphenylpyruvate dioxygenase enzyme, as it was initially determined (SEQ ID NO: 2). Translation start and stop codons are underlined. Selected restriction sites are indicated.

FIG. 3 presents the amino acid sequence comparison between full-length p-hydroxyphenylpyruvate dioxygenases from Arabidopsis thaliana (SEQ ID NO: 15) and Zea mays (SEQ ID NO: 11) and the p-hydroxyphenylpyruvate dioxygenase enzymes derived from human (SEQ ID NO: 6, GenBank Acc. No. U29895), pig (SEQ ID NO: 7, GenBank Acc. No. D13390), mouse (SEQ ID NO: 8, GenBank Acc. No. D29987) and rat (SEQ ID NO: 9, GenBank Acc. No. M18405). Asterisks indicate amino acid residues that are conserved across all six species. This figure was created using the Pileup program of GCG (Program Manual for the Wisconsin Package, Version 9.0-OpenVMS, December 1996, Genetics Computer Group, 575 Science Drive, Madison, Wis., U.S.A. 53711).

FIG. 4 is a diagram describing the construction of the intermediate plasmid vector pT7BlueR+PDO1.

FIG. 5 is a diagram describing the construction of E. coli expression vector pE24CP1.

Applicants have provided a sequence listing in conformity with “Rules for the Standard Representation of Nucleotide and Amino Acid Sequences in Patent Applications” (Annexes I and II to the Decision of the President of the EPO, published in Supplement No. 2 to OJ EPO, 12/1992) and with 37 C. F. R. 1.821-1.825 and Appendices A and B (“Requirements for Application Disclosures Containing Nucleotides and/or Amino Acid Sequences”).

SEQ ID NO: 1 presents a partial nucleic acid sequence of an expressed sequence tag (EST) bearing GenBank Accession No. T92052 obtained from an Arabidopsis thaliana cDNA library. This sequence was contained in clone 91B13T7 of the library.

SEQ ID NO: 2 presents the initial determination of the nucleic acid sequence and the deduced amino acid sequence of a cDNA encoding a full-length form of Arabidopsis thalianap-hydroxyphenylpyruvate dioxygenase enzyme, as contained in plasmid pGBPPD2.

SEQ ID NO: 3 presents the initially deduced amino acid sequence encoded by a cDNA for Arabidopsis thaliana p-hydroxyphenylpyruvate dioxygenase enzyme.

SEQ ID NOS: 4 and 5 present the nucleotide sequences of a pair of complementary oligonucleotides (CAM 32 and CAM 33, respectively) used to facilitate subcloning and expression of the gene encoding p-hydroxyphenyl-pyruvate dioxygenase without the chloroplast transit sequence.

SEQ ID NO: 6 presents the amino acid sequence of p-hydroxyphenyl-pyruvate dioxygenase enzyme derived from human (GenBank Acc. No. U29895).

SEQ ID NO: 7 presents the amino acid sequence of p-hydroxyphenyl-pyruvate dioxygenase enzyme derived from pig (GenBank Acc. No. D1 3390).

SEQ ID NO: 8 presents the amino acid sequence of p-hydroxyphenyl-pyruvate dioxygenase enzyme derived from mouse (GenBank Acc. No. D29987).

SEQ ID NO: 9 presents the amino acid sequence of p-hydroxyphenyl-pyruvate dioxygenase enzyme derived from rat (GenBank Acc. No. M18405).

SEQ ID NO: 10 presents the nucleic acid sequence and deduced amino acid sequence of the cloned cDNA encoding the Zea mays p-hydroxyphenylpyruvate dioxygenase enzyme, as contained in plasmid pMPDO.

SEQ ID NO: 11 presents the deduced amino acid sequence of the cloned cDNA encoding the Zea mays p-hydroxyphenylpyruvate dioxygenase enzyme, as contained in plasmid pMPDO.

SEQ ID NO: 12 presents the nucleic acid sequence and the deduced amino acid sequence of the truncated form of Arabidopsis thaliana p-hydroxyphenyl-pyruvate dioxygenase enzyme as contained in pE24CP1.

SEQ ID NO: 13 presents the deduced amino acid sequence of the truncated form of Arabidopsis thaliana p-hydroxyphenylpyruvate dioxygenase enzyme as contained in pE24CP1.

SEQ ID NO: 14 presents the revised nucleic acid sequence and the deduced amino acid sequence of the cloned cDNA encoding the full-length Arabidopsis thaliana p-hydroxyphenylpyruvate dioxygenase enzyme, as contained in plasmid pGBPPD2.

SEQ ID NO: 15 presents the revised amino acid sequence deduced from the cDNA for the full length Arabidopsis thaliana p-hydroxyphenylpyruvate dioxygenase enzyme.

SEQ ID NO: 16 presents the nucleic acid sequence determined from a portion of a cDNA from Vernonia galamenensis, as contained in clone vsl .pk0015.b2.

### DETAILS OF THE INVENTION

### BIOLOGICAL DEPOSITS

The following biological materials have been deposited under the terms of the Budapest Treaty at American Type Culture Collection (ATCC), 12301 Parklawn Drive, Rockville, Md. 20852, and bear the following accession numbers:

| Depositor Identification | | Int'l. Depository | |
|---|---|---|---|
| Host Strain | Plasmid | Accession Number | Date of Deposit |
| E. coli BL21(DE3) | pE24CP1 | ATCC 98083 | Jun. 25, 1996 |
| N/A | pGBPPD2 | ATCC 97622 | Jun. 25, 1996 |
| N/A | pMPDO | ATCC 209120 | Jun. 12, 1997 |

Definitions

In the context of this disclosure, a number of terms shall be utilized. As used herein, the term “nucleic acid” refers to a large molecule which can be single-stranded or double-stranded, composed of monomers (nucleotides) containing a sugar, phosphate and either a purine or pyrimidine. A “nucleic acid fragment” is a portion of a given nucleic acid molecule. As used herein, “DNA” (deoxyribonucleic acid) is the genetic material, whereas “RNA” (ribonucleic acid) is involved in the transfer of the information encoded by the DNA into proteins and polypeptides. A “genome” is the entire body of genetic material contained in each cell of an organism. The term “nucleotide sequence” refers to a polymer of DNA or RNA which can be single- or double-stranded, optionally containing synthetic, non-natural or altered nucleotide bases capable of incorporation into DNA or RNA polymers.

As used herein, “essentially similar” refers to DNA sequences that may involve base changes that do not cause a change in the encoded amino acid or which involve base changes which may alter one or more amino acids, but do not affect the functional properties of the protein encoded by the DNA sequence. It is therefore understood that the invention encompasses more than the specific exemplary sequences. Modifications to the sequence, such as deletions, insertions, or substitutions in the sequence which produce “silent changes” (i.e., those that do not substantially affect the functional properties of the resulting protein molecule) are also contemplated. For example, alteration(s) in the gene sequence which reflects the degeneracy of the genetic code, or which result in the production of a chemically equivalent amino acid at a given site, are contemplated; thus, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a biologically equivalent product. Nucleotide changes which result in alteration of the N-terminal and C-terminal portions of the protein molecule would also not be expected to alter the activity of the protein. In some cases, it may in fact be desirable to make mutants of the sequence in order to study the effect of alteration on the biological activity of the protein. Each of the proposed modifications is well within the routine skill in the art, as is determination of retention of biological activity of the encoded products. Moreover, the skilled artisan recognizes that “essentially similar” sequences encompassed by this invention are also defined by their ability to hybridize, under stringent conditions (0.1X SSC, 0.1% SDS, 65° C.), with the sequences exemplified herein.

“Gene” refers to a nucleic acid fragment that encodes a specific protein, including regulatory sequences preceding (5′ non-coding) and following (3′ non-coding) the coding region. “Native” gene refers to the gene as found in nature with its own regulatory sequences. “Chimeric” gene refers to a gene comprising heterogeneous regulatory and coding sequences. “Endogenous” gene refers to the native gene normally found in its natural location in the genome. A “foreign” gene refers to a gene not normally found in the host organism but that is introduced by gene transfer.

“Coding sequence” refers to a DNA sequence that codes for a specific protein and excludes the non-coding sequences.

“Initiation codon” and “termination codon” refer to a unit of three adjacent nucleotides in a coding sequence that specifies initiation and termination, respectively, of protein synthesis (mRNA translation). “Open reading frame” refers to the amino acid sequence encoded between translation initiation and termination codons of a coding sequence.

“RNA transcript” refers to the product resulting from RNA polymerase-catalyzed transcription of a DNA sequence. When the RNA transcript is a perfect complementary copy of the DNA sequence, it is referred to as the primary transcript or it may be a RNA sequence derived from posttranscriptional processing of the primary transcript. “Messenger RNA” (mRNA) refers to RNA that can be translated into protein by the cell. “cDNA” refers to a double-stranded DNA, one strand of which is complementary to and derived from mRNA by reverse transcription. “Sense RNA” refers to RNA transcript that includes the mRNA.

As used herein, “regulatory sequences” are nucleotide sequences that control the transcription or expression of a coding sequence located upstream (5′), within, or downstream (3′) to the coding sequence, act in conjunction with the protein biosynthetic apparatus of the cell and include promoters, translation leader sequences, transcription termination sequences, and polyadenylation sequences.

“Promoter” refers to a DNA sequence in a gene, usually upstream (5′) to its coding sequence, which controls the expression of the coding sequence by providing the recognition for RNA polymerase and other factors required for proper transcription. A promoter may also contain DNA sequences that are involved in the binding of protein factors which control the effectiveness of transcription initiation in response to physiological or developmental conditions. In the case of eukaryotic organisms, it may also contain enhancer elements.

An “enhancer element” is a DNA sequence which can stimulate promoter activity. It may be an innate element of the promoter or a heterologous element inserted to enhance the activity level and tissue-specificity of a promoter. “Constitutive promoters” refer to those enhancer elements that direct gene expression in all tissues and at all times. “Organ-specific” or “development-specific” promoters as referred to herein are those that direct gene expression almost exclusively in specific organs, such as leaves or seeds, or at specific development stages in an organ, such as in early or late embryogenesis, respectively.

The term “operably linked” refers to nucleic acid sequences on a single nucleic acid molecule which are associated so that the function of one is affected by the other. For example, a promoter is operably linked with a structural gene (i.e., a gene encoding p-hydroxyphenylpyruvate dioxygenase, as disclosed herein) when it is capable of affecting the expression of that structural gene (i.e., that the structural gene is under the transcriptional control of the promoter).

The term “expression”, as used herein, is intended to mean the production of the protein product encoded by a gene. More particularly, “expression” refers to the transcription and stable accumulation of the sense RNA (mRNA) derived from the nucleic acid fragment(s) of the invention that, in conduction with the protein apparatus of the cell, results in altered levels of protein product.

“Overexpression” refers to the production of a gene product in transgenic organisms that exceeds levels of production in normal or non-transformed organisms. “Altered levels” refers to the production of gene product(s) in transgenic organisms in amounts or proportions that differ from that of normal or non-transformed organisms. “Facilitating expression” refers to steps and conditions for culturing host cells containing the desirable gene to yield an increased production of the enzyme. For example, addition of a chemical inducer specific to the particular promoter operably linked to the gene facilitates expression of the encoded enzyme. This is measured relative to the production levels of an untreated gene.

The “3′ non-coding sequences” refers to the DNA sequence portion of a gene that contains a polyadenylation signal and any other regulatory signal capable of affecting mRNA processing or gene expression. The polyadenylation signal is usually characterized by affecting the addition of polyadenylic acid tracts to the 3′ end of the mRNA precursor.

The “translation leader sequence” refers to that DNA sequence portion of a gene between the promoter and coding sequence that is transcribed into RNA and is present in the fully processed mRNA upstream (5′) of the translation start codon. The translation leader sequence may affect processing of the primary transcript to mRNA, mRNA stability, or translation efficiency.

“Transformation” herein refers to the transfer of a foreign gene into the genome of a host organism and its genetically stable inheritance. Bacterial transformation can proceed by any of several methods well known in the art, including calcium chloride-mediated transformation and electroporation. Examples of methods of plant transformation include Agrobacterium-mediated transformation and particle-accelerated or “gene gun” transformation technology (U.S. Pat. No. 4,945,050).

“Host cell” refers to the cell that is transformed with the introduced genetic material.

“Plasmid vector” refers to a double-stranded, closed circular, extra-chromosomal DNA molecule.

“Tolerant” or “tolerance” refers to a condition whereby a cell or an organism is able to withstand the effect of application of a compound or composition at a concentration or application rate that causes a demonstrable effect in or against cells or organisms that are not tolerant. For example, the growth or survival of a plant that is tolerant to application of a herbicidal compound or composition will be less affected than the growth or survival of a plant that is not tolerant to application of the herbicidal compound or composition.

Cloning of Plant Genes Encoding p-Hydroxyphenylpyruvate Dioxygenase

The p-hydroxyphenylpyruvate dioxygenases from plants are a promising new class of targets for new herbicidal compounds. In order to be able to study this enzyme in detail, and to have available supplies of enzyme for inhibitor screening, cDNA clones encoding plant p-hydroxyphenylpyruvate dioxygenases were identified. These nucleic acid fragments are useful for the production of their encoded enzymes, for isolation of clones from additional plant sources that encode other p-hydroxyphenylpyruvate dioxygenase enzymes, and for understanding the biochemical and structural properties of these enzymes.

Nucleic acid fragments comprising nucleotide sequences that encode different forms of the enzyme p-hydroxyphenylpyruvate dioxygenase from the plant Arabidopsis thaliana have now been isolated. Subsequently, these nucleotide sequences were expressed in E. coli cells and shown to direct the synthesis of plant p-hydroxyphenylpyruvate dioxygenase enzymes.

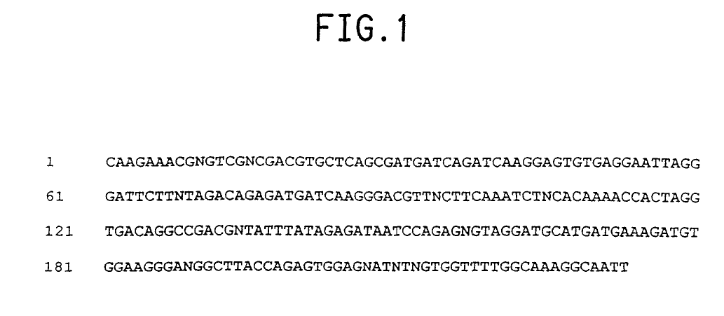

An automated search of nucleotide sequences contained in a database representing an Arabidopsis cDNA library for sequences homologous to other known, non-plant p-hydroxyphenylpyruvate dioxygenase genes revealed the plasmid cDNA clone 91B13T7. This cDNA was obtained from the Arabidopsis Seed Stock Center at Ohio State University. Plasmid DNA suitable for nucleotide sequence determination was prepared and the nucleotide sequence of the plasmid insert was determined. The resulting sequence was not interpretable, suggesting possible contamination of the plasmid sample by an extraneous nucleic acid. This assumption was confirmed by digesting the plasmid DNA sample with restriction enzymes and separating the resulting nucleic acid fragments by agarose gel electrophoresis. This analysis revealed the presence of nucleic acid fragments that could not be derived from the plasmid carrying the putative p-hydroxyphenyl-pyruvate dioxygenase fragment. Furthermore, a search of the publically available nucleic acid sequence databases revealed that the Arabidopsis thaliana sequence reported for cDNA clone 91B13T7 corresponded to a truncated cDNA (FIG. 1). Based on publically available mammalian cDNA sequence information for p-hydroxyphenylpyruvate dioxygenase, the minimum length expected for a cDNA encoding a complete p-hydroxyphenylpyruvate dioxygenase enzyme is 1 kb (Table 1).

TABLE 1
Predicted cDNA Length for Sequences
Encoding p-Hydroxyphenylpyruvate Dioxygenase

| | Amino Acid | |
|---|---|---|
| Organism | Residues | Minimum cDNA (kb) |
| Human | 392 | 1.176 |
| Pig | 392 | 1.176 |
| Pseudomonas sp. | 357 | 1.071 |

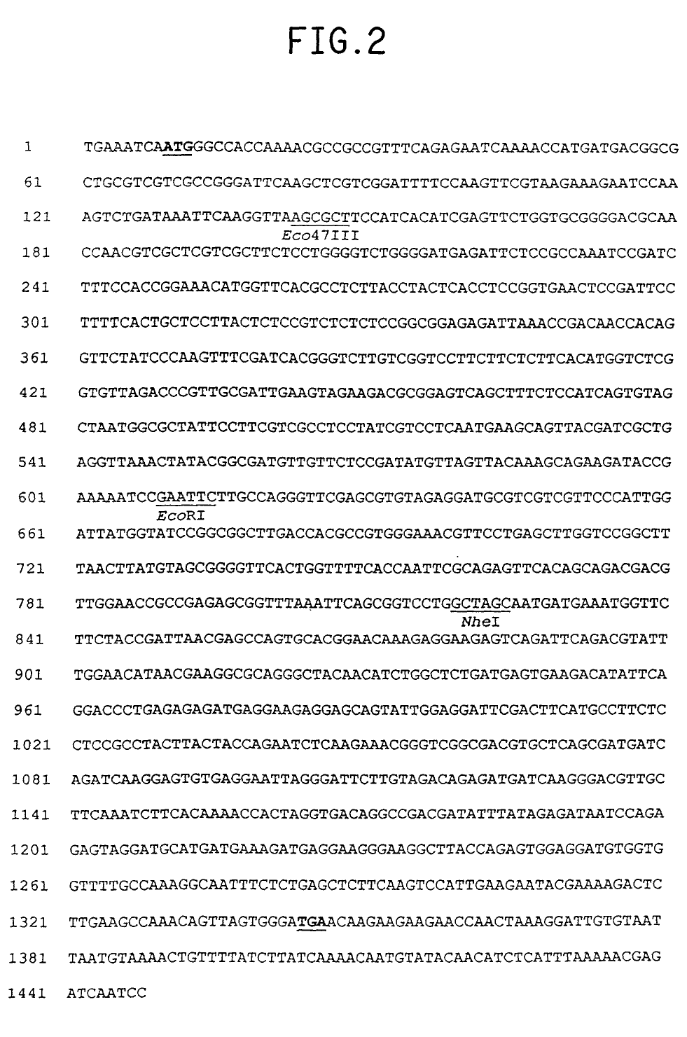

Therefore, based on the expected length of a cDNA capable of encoding a functional p-hydroxyphenylpyruvate dioxygenase, the Arabidopsis thaliana sequence obtained from the public database was insufficient to encode a full-length, active p-hydroxyphenylpyruvate dioxygenase enzyme. Therefore, a cDNA with the capacity to encode a full-length enzyme Arabidopsis thaliana was cloned, as described herein. A 400 bp segment of the insert of plasmid 91B13T7 was liberated by digestion with restriction enzymes and used to screen a cDNA library prepared from norflurazon-treated Arabidopsis thaliana seedlings (Scolnik, P. A., and Bartley, G. E. (1994) Plant Physiol. 104:1469-1470). Several clones showing positive hybridization to this probe were sequenced. The initial determination of the sequence of the longest cDNA clone obtained from this effort is shown in FIG. 2 and in SEQ ID NO: 2. During the course of subsequent work with this clone it became necessary to confirm certain features of the sequence. A corrected sequence of this cDNA is presented in SEQ ID NO: 12.

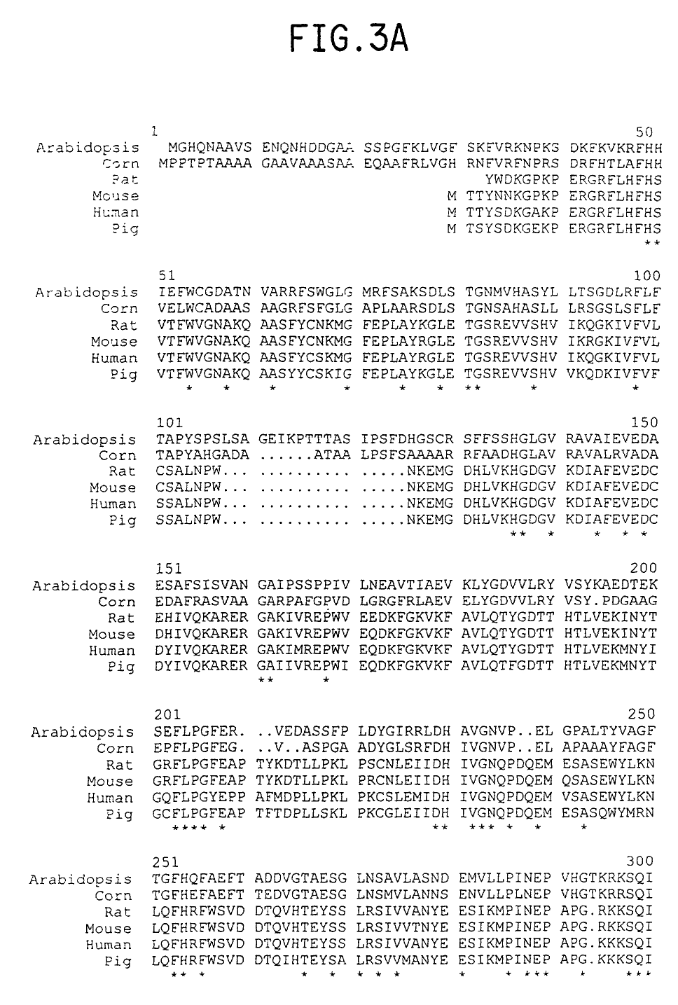

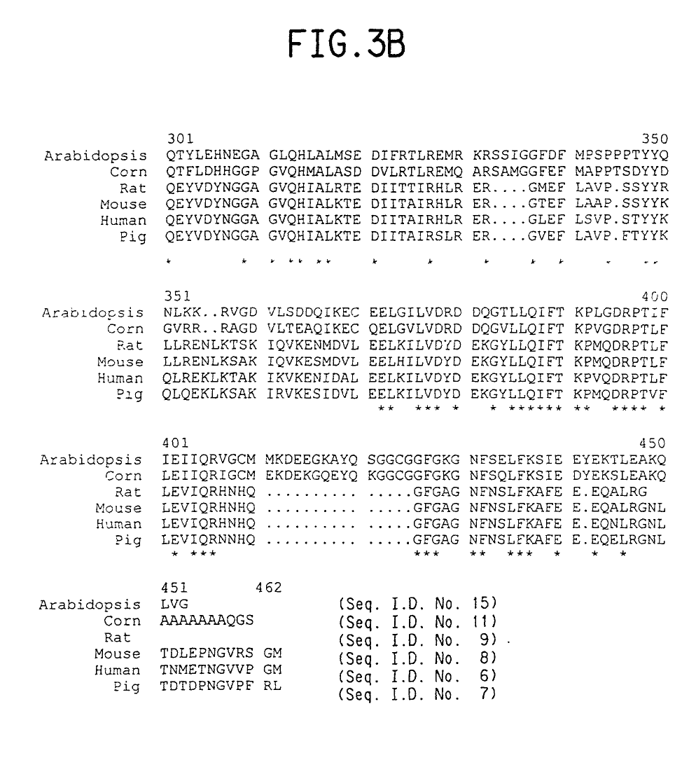

The sequence reported in FIG. 2 indicates that this cDNA has the capacity to encode a protein of MW 48,841 which, as shown in FIG. 3, has a high level of homology to p-hydroxyphenylpyruvate dioxygenase enzymes from other eukaryotes.

A cDNA capable of encoding a full-length p-hydroxyphenylpyruvate dioxygenase has also been obtained from corn. This cDNA, contained in plasmid pMPDO, was identified in a corn cDNA library using an approximately 900 base pairs portion of the Arabidopsis cDNA as a probe. The predicted amino acid sequence that is encoded by the corn cDNA is also compared to p-hydroxyphenylpyruvate dioxygenase enzymes from other eukaryotes in FIG. 3.

A cDNA library was prepared from messenger RNA isolated from developing seeds of Vernonia galamenensis. Random sequencing of the clones contained in the library identified a probable clone, designated vs1.pk0015.b2, for the p-hydroxyphenylpyruvate dioxygenase from this plant. The 513 bp expressed sequence tag (EST) is presented in SEQ ID NO: 16.

Expression of the Arabidopsis thaliana cDNA Encoding p-Hydroxyphenylpyruvate Dioxygenase in E. coli

The nucleic acid fragments of the instant invention encoding a plant p-hydroxyphenylpyruvate dioxygenase enzymes can be operably linked to suitable regulatory sequences, thereby creating chimeric genes that can be used to direct expression of the enzyme in transgenic organisms. These transgenic organisms include, but are not limited to: plants (Plant Molecular Biology; Croy, R. R. D., Ed.; Bios Scientific Publishers; 1993); microorganisms, including Escherichia coli (Gold, L. (1990) Methods in Enzymology 185:11), Bacillus subtilis (Henner, D. J. (1990) Methods in Enzymology 185:199), yeast (Gellissen, G., et al. (1992) Antonie Leeuwenhoek 62:79), and fungi, including members of the genus Aspergillus (Devchand, M. and Gwynne, D. I. (1991) J. Biotechnol. 17:3); and insect cells containing recombinant baculoviruses (Lukow, V. A. and Summers, M. D. (1988) Bio/Technology 6:47).

One skilled in the art can isolate the coding sequences from the fragments of the invention by using or creating sites for restriction endonucleases, as described in Sambrook, J., et al.((989) Molecular Cloning, A Laboratory Manual, 2nd ed.; Cold Spring Harbor Laboratory Press; hereinafter “Maniatis”). Alternatively, polymerase chain reaction (PCR) techniques can be employed to isolate and/or modify the fragments of the invention (Newton, C. R. and Graham, A. (1994) PCR; Bios Scientific Publishers).

Arabidopsis p-hydroxyphenylpyruvate dioxygenase was expressed in E. coli under control of a T7 promoter in a strain expressing T7 RNA polymerase (Studier, F. W., et al. (1990) Methods in Enzymology 185:60). Promoters other than T7 are commonly used in expression vectors and could be substituted for protein expression in E. coli. Examples of alternative promoters include, but are not limited to, trp (Yansura, D. G. and Henner, D. J. (1990) Methods in Enzymology 185:54), P_{L} (Remaut, E. et al. (1981) Gene 15:81), tac (Amann, E. et al. (1983) Gene 25:167), trc (Amann, E. et al. (1988) Gene 69:301), and promoters such as lacUV5, lₚₚ, P_{R}, and hybrid and tandem promoters constructed to combine specific features to increase strength or regulation capacity (Balbas, P. and Bolivar, F. (1990) Methods in Enzymology 185:14).

Biochemical Evidence of Enzymatic Function

The enzyme p-hydroxyphenylpyruvate dioxygenase catalyzes the reaction of p-hydroxyphenylpyruvate with molecular oxygen to give homogentisate and CO₂. The enzyme can be assayed by measuring oxygen utilization (Hager, S. E., et al. (1957) J. Biol. Chem. 225:935-947), CO₂ release or homogentisate production from radioactive labeled p-hydroxyphenylpyruvate (Lindblad, B. (1971) Clin. Chem. Acta 34:113-121), loss of the p-hydroxyphenylpyruvate (Lin, E. C. C. et al. (1958) J. Biol. Chem. 233:668-673), or formation of homogentisate using a calorimetric assay (Fellman, J. H. et al. (1972) Biochim. Biophys. Acta 284:90-100) or UV detection following HPLC or a similar chromatographic separation technique. The activity of p-hydroxyphenylpyruvate dioxygenase may also be measured in a coupled assay in which the initial product, homogentisate, is oxidized by homogentisate dioxygenase; formation of maleylacetoacetate determined by measuring absorbance at 330 nm (Fernandez-Caeñon, J. M. and Peñalva, M. A. (1997) Anal. Biochem. 245:218-221).

An alternative to any of the kinetic assays for p-hydroxyphenylpyruvate dioxygenase is an end-point or fixed-time assay. The procedure is based on the conversion of unconverted substrate, p-hydroxyphenylpyruvate to its enediol tautomer by tautomerase in the presence of borate ions and measurement of the characteristic 308 nm peak of the tautomer (Lin, E. C. C. et al. (1958) J. Biol. Chem. 233:668-673). The procedure involves the addition of enough p-hydroxyphenylpyruvate dioxygenase to consume ˜80% of the organic substrate over 1 hour in 200 μL of assay buffer, which in this case is a 50 mM Tris, pH 7.4, 0.10 mM p-hydroxyphenylpyruvic acid, 1.75 mM ascorbate and 1.25 mM EDTA. After 1 hr the reaction is quenched by the addition of 100 μL of 0.8 M borate, pH 7.3, containing 1000 ppb of a p-hydroxyphenylpyruvate dioxygenase inhibitor and 0.25 μL of 6.1 mg/mL of tautomerase. The absorbance at 308 nm is read after a 30 min incubation and is stable thereafter for 2 hr. The advantage of this assay over the kinetic procedure is that the p-hydroxyphenylpyruvate dioxygenase is not required to oxidize the substrate in the presence of high concentrations of borate, a condition that might interfere with the mode of action of inhibitors. Furthermore the assay produces essentially a stable binary indication of p-hydroxyphenylpyruvate dioxygenase inhibition, and is well-suited for applications which require a high-throughput of samples and assays.

The enzyme encoded by the nucleic acid fragments and overexpressed in E. coli can be extracted in any conventional buffer used for extracting soluble plant enzymes. Although a large amount of an overexpressed protein is often insoluble, the amount that is soluble represents can represent as much as 50% of the total soluble protein. Soluble overexpressed protein has high p-hydroxyphenylpyruvate dioxygenase activity and is easily extracted. Likewise, it may be possible to resolubilize an insoluble overexpressed protein in an active form under appropriate conditions, since addition of sarkosyl (sodium N-lauroylsarcosinate) to the extraction buffer appeared to increase the amount of the overexpressed protein extracted. For optimum activity, a reducing agent such as ascorbate or reduced glutathione should be present as well as a source a ferrous ion.

An overexpressed enzyme can be assayed using all the techniques described above for measuring p-hydroxyphenylpyruvate dioxygenase activity, while only the techniques using labeled p-hydroxyphenylpyruvate can be used to measure activity in crude plant extracts. Therefore, the availability of an overexpressed enzyme greatly facilitates the development of high capacity screens to identify inhibitors of the enzyme. Potential inhibitors are evaluated for their capacity to reduce the rate of the reaction of the enzyme, resulting in reduced oxygen uptake and CO₂ release, and lower rates of formation of homogentisate and loss of p-hydroxyphenylpyruvate. Applicants have demonstrated that at least one of the instant nucleic acid fragments can be overexpressed in E. coli cells, resulting in production of a protein that catalyzes the conversion of p-hydroxy-phenylpyruvate to homogentisate with the release of CO₂. Furthermore, it has been shown that this activity is inhibited by commercial herbicides known to inhibit p-hydroxyphenylpyruvate dioxygenase. Finally, an overexpressed enzyme can be used in a high capacity assay to identify compounds that inhibit the enzymatic activity of p-hydroxyphenylpyruvate dioxygenase. Such compounds may serve as herbicides.

Preparation of Plants Tolerant to Inhibitors of p-Hydroxyphenylpyruvate Dioxygenase

This invention embodies plants which are resistant or at least tolerant to herbicides that target the p-hydroxyphenylpyruvate dioxygenase enzyme at levels which are normally inhibitory to the naturally occurring p-hydroxyphenylpyruvate dioxygenase enzyme. This altered p-hydroxyphenylpyruvate dioxygenase activity is conferred by (1) overexpression of the wild-type p-hydroxyphenylpyruvate dioxygenase enzyme, or (2) expression of a DNA molecule encoding a herbicide-tolerant enzyme. The said enzyme may be a modified form of an p-hydroxyphenylpyruvate dioxygenase enzyme that occurs naturally in a eukaryote or prokaryote, or a modified form of an p-hydroxyphenylpyruvate dioxygenase enzyme that naturally occurs in a plant, or a herbicide tolerant enzyme that naturally occurs in a prokaryote (Duke et al. Herbicide Resistant Crops; Lewis: Boca Raton; 1994). An effective amount of gene expression to render the cells of the plant tissue substantially tolerant to the herbicide depends on whether the gene codes for an unaltered p-hydroxyphenylpyruvate dioxygenase gene or a mutant or altered form of the gene that is less sensitive to the herbicides. Expression of an unaltered plant p-hydroxyphenylpyruvate dioxygenase gene in an effective amount is that amount that provides for a 2- to 10-fold increase in herbicide tolerance. Plants encompassed by the invention include monocotyledoneous and dicotyledoneous plants. Preferred are those plants which would be potential targets for p-hydroxyphenylpyruvate dioxygenase-inhibiting herbicides, particularly agronomically important crops such as maize and other cereal crops.

Increased levels of expression of p-hydroxyphenylpyruvate dioxygenase activity, from two to ten or more times the natively expressed amount, would be sufficient to overcome growth inhibition caused by the herbicide. Plants containing such altered p-hydroxyphenylpyruvate dioxygenase enzyme activity can be obtained by direct selection in plants. This method is known in the art. See, e.g., U.S. Pat. No. 5,162,602, U.S. Pat. No. 4,761,373, and references cited therein.

Overexpression of p-hydroxyphenylpyruvate dioxygenase also can be accomplished by stably transforming a host plant cell with a chimeric DNA molecule comprising a promoter capable of driving expression of an associated coding sequence in a plant cell and operably linked to a homologous or heterologous coding sequence encoding p-hydroxyphenylpyruvate dioxygenase. A “homologous” p-hydroxyphenylpyruvate dioxygenase gene is isolated from an organism taxonomically identical to the target plant cell, whereas a “heterologous” p-hydroxyphenylpyruvate dioxygenase gene is obtained from an organism taxonomically distinct from the target plant.

The expression of foreign genes in plants is well-established (De Blaere et al., (1987) Meth. Enzymol 143:277-291). Promoters utilized to drive gene expression in transgenic plants or plant cells (i.e., those capable of driving expression of the associated coding sequences such as p-hydroxyphenylpyruvate dioxygenase in plant cells, include those directing the 19S and 35S transcripts in Cauliflower mosaic virus (Odell et al., (1985) Nature 313:810-812; Hull et al., (1987) Virology 86:482-493), small subunit of ribulose 1,5-bisphosphate carboxylase (Morelli et al., (1985) Nature 315:200-204; Broglie et al., (1984) Science 224:838-843; Hererra-Estrella et al., (1984) Nature 310:115-120; Coruzzi et al., (1984) EMBO J. 3:1671-1679; Faciotti et al., (1985) Bio/Technology 3:241 and chlorophyll a/b binding protein (Lamppa et al., (1986) Nature 316:750-752); nopaline synthase promoters (Depicker et al. (1982) J. Mol. App. Genet. 1:561-573; An et al. (1990) Plant Cell 2:225-233). The chimeric DNA construct(s) of the invention may contain multiple copies of a promoter or multiple copies of the p-hydroxyphenylpyruvate dioxygenase coding sequences. In addition, the construct(s) may include coding sequences for selectable markers and coding sequences for other peptides such as signal or transit peptides. The preparation of such constructs is within the ordinary level of skill in the art. Resistance to inhibitors of the plant carotenoid biosynthesis pathway, which is also targeted by p-hydroxyphenylpyruvate dioxygenase inhibitors, has been achieved by expressing a bacterial gene encoding phytoene desaturase driven by the CaMV promoter (Misawa et al., (1994) Plant. J. 4:481-490).

Transit peptides may be fused to the p-hydroxyphenylpyruvate dioxygenase coding sequence in the chimeric DNA constructs of the invention to direct transport of the expressed p-hydroxyphenylpyruvate dioxygenase enzyme to the desired site of action. Examples of transit peptides include the chloroplast transit peptides such as those described in Von Heijne et al., (1991) Plant Mol. Biol. Rep. 9:104-126; Mazur et al., (1987) Plant Physiol. 85:1110; Vorst et al., (1988) Gene 65:59; and mitochondrial transit peptides such as those described in Boutry et al., (1987) Nature 328:340-342.

It is envisioned that the introduction of enhancers or enhancer-like elements into other promoter constructs will also provide increased levels of primary transcription to accomplish the invention. These would include viral enhancers such as that found in the 35S promoter (Odell et al., (1988) Plant Mol. Biol. 10:263-272), enhancers from the opine genes (Fromm et al., (1989) Plant Cell 1:977-984), or enhancers from any other source that result in increased transcription when placed into a promoter operably linked to the nucleic acid fragment of the invention.

Introns isolated from the maize Adh-1 and Bz-1 genes (Callis et al., (1987) Genes Dev. 1:1183-1200), and intron 1 and exon 1 of the maize Shrunken-1 (sh-1) gene (Maas et al., (1991) Plant Mol. Biol. 16:199-207) may also be of use to increase expression of introduced genes. Results with the first intron of the maize alcohol dehydrogenase (Adh- 1) gene indicate that when this DNA element is placed within the transcriptional unit of a heterologous gene, mRNA levels can be increased by 6.7-fold over normal levels. Similar levels of intron enhancement have been observed using intron 3 of a maize actin gene (Luehrsen, K. R. and Walbot, V., (1991) Mol. Gen. Genet. 225:81-93). Enhancement of gene expression by Adh1 intron 6 (Oard et al., (1989) Plant Cell Rep 8:156-160) has also been noted. Exon1 and intron1 of the maize sh-1 gene have been shown to individually increase expression of reporter genes in maize suspension cultures by 10 and 100-fold, respectively. When used in combination, these elements have been shown to produce up to 1000-fold stimulation of reporter gene expression (Maas et al., (1991) Plant Mol. Biol. 16:199-207).

Any 3′ non-coding region capable of providing a polyadenylation signal and other regulatory sequences that may be required for proper expression can be used to accomplish the invention. This would include the 3′ end from any storage protein such as the 3′ end of the 10kd, 15kd, 27kd and alpha zein genes, the 3′ end of the bean phaseolin gene, the 3′ end of the soybean β-conglycinin gene, the 3′ end from viral genes such as the 3′ end of the 35S or the 19S cauliflower mosaic virus transcripts, the 3′ end from the opine synthesis genes, the 3′ ends of ribulose 1,5-bisphosphate carboxylase or chlorophyll a/b binding protein, or 3′ end sequences from any source such that the sequence employed provides the necessary regulatory information within its nucleic acid sequence to result in the proper expression of the promoter/coding region combination to which it is operably linked. There are numerous examples in the art that teach the usefulness of different 3′ non-coding regions (for example, see Ingelbrecht et al., (1989) Plant Cell 1:671-680).

Various methods of introducing a DNA sequence (i.e., of transforming) into eukaryotic cells of higher plants are available to those skilled in the art (see EPO publications 0 295 959 A2 and 0 138 341 A1). Such methods include high-velocity ballistic bombardment with metal particles coated with the nucleic acid constructs (see Klein et al., (1987) Nature (London) 327:70-73, and see U.S. Pat. No. 4,945,050), as well as those based on transformation vectors based on the Ti and Ri plasmids of Agrobacterium spp., particularly the binary type of these vectors. Ti-derived vectors transform a wide variety of higher plants, including monocotyledonous and dicotyledonous plants, such as soybean, cotton and rape seed (Pacciotti et al., (1985) Bio/Technology 3:241; Byrne et al., (1987) Plant Cell, Tissue and Organ Culture 8:3; Sukhapinda et al., (1987) Plant Mol. Biol. 8:209-216; Lorz et al., (1985) Mol. Gen. Genet. 199:178-182; Potrykus et al., (1985) Mol. Gen. Genet. 199:183-188).

Other transformation methods are available to those skilled in the art, such as direct uptake of foreign DNA constructs (see EPO publication 0 295 959 A2), and techniques of electroporation (see Fromm et al., (1986) Nature (London) 319:791-793). Once transformed, the cells can be regenerated by those skilled in the art. Also relevant are several recently described methods of introducing nucleic acid fragments into commercially important crops, such as rapeseed (see De Block et al., (1989) Plant Physiol. 91:694-701), sunflower (Everett et al., (1987) Bio/Technology 5:1201-1204), soybean (McCabe et al., (1988) Bio/Technology 6:923-926; Hinchee et al., (1988) Bio/Technology 6:915-922; Chee et al., (1989) Plant Physiol. 91:1212-1218; Christou et al., (1989) Proc. Natl. Acad. Sci USA 86:7500-7504; EPO Publication 0 301 749 A2), and corn (Gordon-Kamm et al., (1990) Plant Cell 2:603-618; and Fromm et al., (1990) Bio/Technology 8:833-839).

Altered p-hydroxyphenylpyruvate dioxygenase enzyme activity may also be achieved through the generation or identification of modified forms of the isolated eukaryotic p-hydroxyphenylpyruvate dioxygenase coding sequence having at least one amino acid substitution, addition or deletion which encodes an altered p-hydroxyphenylpyruvate dioxygenase enzyme resistant to a herbicide that inhibits the unaltered, naturally occurring form. Genes encoding such enzymes can be obtained by numerous strategies known in the art. A first general strategy involves direct or indirect mutagenesis procedures on microbes (e.g., E. coli, S. cerevisiae (Miller, (1972) Experiments in Molecular Genetics, Cold Spring Harbor Laboratory, Cold Spring Harbor, N.Y.; Davis et al., (1980) Advanced Bacterial Genetics, Cold Spring Harbor Laboratory, Cold Spring Harbor, N.Y.; Sherman et al., (1983) Methods in Yeast Genetics, Cold Spring Harbor Laboratory, Gold Spring Harbor N.Y.; and U.S. Pat. No. 4,975,374) and cyanobacteria (Bryant, The Molecular Biology of Cyanobacteria; Kluwer Academic Publishers: Boston, 1995). A second method of obtaining mutant herbicide-resistant alleles of the eukaryotic p-hydroxyphenylpyruvate dioxygenase enzyme involves direct selection in plants. For example, the effect of inhibitors on the growth of plants such as Arabidopsis, soybean, or maize may be determined by plating seeds sterilized by art-recognized methods on plates on a simple minimal salts medium containing increasing concentrations of the inhibitor. The lowest dose at which significant growth inhibition can be reproducibly detected is used for subsequent experiments. Mutagenesis of plant material may be utilized to increase the frequency at which resistant alleles occur in the selected population. Mutagenized seed material can be derived from a variety of sources, including chemical or physical mutagenesis or seeds, or chemical or physical mutagenesis or pollen (Neuffer, In Maize for Biological Research. Sheridan, ed. Univ. Press, Grand Forks, N. Dak., pp. 61-64 (1982)), which is then used to fertilize plants and the resulting M1mutant seeds collected. Typically, for Arabidopsis, M2 seeds (i.e., progeny seeds of plants grown from seeds mutagenized with chemicals, such as ethyl methane sulfonate, or with physical agents, such as gamma rays or fast neutrons) are plated at densities of up to 10,000 seeds/plate (10 cm diameter) on minimal salts medium containing an appropriate concentration of inhibitor. Seedlings that continue to grow and remain green 7-21 days after plating are transplanted to soil and grown to maturity and seed set. Progeny of these seeds are tested for resistance to the herbicide. If the resistance trait is dominant, plants whose seed segregate 3: 1 (resistant:sensitive) are presumed to have been heterozygous for the resistance at the M2 generation. Plants that give rise to all resistant seed are presumed to have been homozygous for the resistance at the M2 generation. Such mutagenesis on intact seeds and screening of their M2 progeny seed can also be carried out on other species, for instance soybean (see, e.g., U.S. Pat. No. 5,084,082). Mutant seeds to be screened for herbicide tolerance can also be obtained as a result of fertilization with pollen mutagenized by chemical or physical means.

### EXAMPLE 1

### Cloning of a cDNA for Arabidopsis thaliana p-Hydroxyphenylpyruvate Dioxygenase

The plasmid containing the Arabidopsis thaliana 91B 13T7 expressed sequence tag (Newman et al., (1994) Plant Physiol 106:1241-1255) was digested with the restriction enzymes BamHI and EcoRI, and the resulting 400 bp fragment was used to screen a lambda phage cDNA library of Arabidopsis thaliana seedlings (Scolnik, P. A. and Bartley, G. E. (1994) Plant Physiol. 104:1469-1470) according to the following protocol.

E. coli KW251 cells were grown overnight in Luria Broth (“LB”) containing 0.2% maltose and 10 mM MgSO₄. Cells were pelleted by centrifugation and resuspended in 10 mM MgSO₄ to an OD₆₀₀ of 0.5. Cell aliquots (0.8 mL) were mixed with 0.1 mL of diluted phage samples and 7 mL of top agarose (0.7% agarose in LB containing 10 mM MgSO₄) at 45° C., and plated onto 150 mm Petri dishes containing LB agar. Phage plaques became visible in 5-7 h, at which point the plates were placed at 4° C.

Phage plaques were transferred to nitrocellulose filters according to standard techniques, and the filters were hybrized to ³²P-radiolabeled probe prepared according to the method of Feinberg and Vogelstein ((1983) Anal. Biochem. 132:6-13), using the hybridization conditions of Berlyn et al.((1989) Proc. Natl. Acad. Sci. 86:4604-4608). After exposure to X-ray film for 48 h, 12 positive plaques were eluted, plated, and hybridized under the same conditions. A total of 9 plaques that retained positive signals in this second round of hybridization were subjected to in vivo excision using the Exassist/SOLR™ system according to the manufacturer's protocol (Stratagene Cloning Systems, La Jolla, Calif.). DNA from the plasmids resulting from in vivo excision of positive plaques was prepared for DNA sequencing using the Wizard Plus™ kit (Promega, Madison, Wis.). Eight of the clones that were sequenced showed strong conservation with available p-hydroxyphenylpyruvate dioxygenase sequences, whereas the remaining clone did not correspond to a p-hydroxyphenylpyruvate dioxygenase. Alignment with known p-hydroxyphenylpyruvate dioxygenase sequences also revealed that two of the clones correspond to 0.3 kbp fragments from the 3′ end of the transcript, and another two to 1.2 kbp fragments from the 5′ end of the transcript. One clone of each was used to assemble a 1.5 kbp cDNA by ligating at the internal NheI restriction site (FIG. 1). The initial determination of the DNA sequence (SEQ ID NO: 2) of the resulting cDNA clone is shown in FIG. 2. Subsequent work with this DNA fragment required confirmation of some of the features of its sequence. Approximately ten nucleotide residues were found to have been listed in error. Thus a corrected sequence for this DNA fragment is listed in SEQ ID NO: 14 and the deduced amino acid sequence is set forth in SEQ ID NO: 15. The revised sequences form the bases for analyses and comparisons reported herein.

### EXAMPLE 2

### Overexpression of the Arabidopsis cDNA in E. coli

The deduced amino acid sequence for Arabidopsis p-hydroxyphenylpyruvate dioxygenase was aligned with the amino acid sequences of p-hydroxyphenylpyruvate dioxygenase from mouse, pig, and Streptomyces avermitilis using the Pileup program of GCG (Program Manual for the Wisconsin Package, Version 8, September 1994, Genetics Computer Group, 575 Science Drive, Madison, Wis., U.S.A. 53711). This analysis suggested an additional 29 amino acid-extension at the amino terminus of the Arabidopsis sequence (positions 1-29, FIG. 3 and SEQ ID NO: 3). This amino-terminal extension was assumed to be a chloroplast transit peptide which would be absent from the mature enzyme. Therefore, removal of the chloroplast transit peptide coding sequence coincided with transfer of the p-hydroxyphenylpyruvate dioxygenase coding sequence from the cloning vector into the expression vector.

The Arabidopsisp-hydroxyphenylpyruvate dioxygenase cDNA was moved from the pBluescript SK-cloning vector (Stratagene, La Jolla, Calif.) to the pET24c(+) expression vector (Novagen, Madison, Wis.) through the intermediate cloning vector pT7BlueR (Novagen). The plasmid pGBPPD2 consists of the Arabidopsisp-hydroxyphenylpyruvate dioxygenase cDNA and the pBluescript SK-cloning vector (Stratagene). The plasmid pE24CP1 consists of the Arabidopsisp-hydroxyphenylpyruvate dioxygenase cDNA, without the putative chloroplast transit peptide DNA sequence, and the pET24c(+) expression vector (Novagen).

The plasmids pGBPPD2 and pT7BlueR (5 μg each) were individually digested with 20 units of Xba I (New England Biolabs, NEB, Beverly, Mass.) and 20 units of Hind III (Gibco BRL, Gaithersburg, Md.) in NEB restriction enzyme buffer 2 supplemented with 100 μg/mL bovine serum albumin at 37° C. for 1.75 h. Digesting pGBPPD2 with the restriction enzymes Xba I and Hind III releases the 5′ and 3′ ends, respectively, of the p-hydroxyphenylpyruvate dioxygenase cDNA from the pBluescript SK-polylinker. Products of the digestion were electro-phoretically separated in a 1 percent agarose gel using TRIS/acetate/EDTA (TAE) buffer and visualized with ethidium bromide staining (Maniatis). Digestion of pGBPPD2 with the two restriction endonucleases resulted in a 2922 bp vector band and 1499 bp p-hydroxyphenylpyruvate dioxygenase cDNA band. Only a 2863 bp band was apparent after digesting pT7BlueR with the two enzymes, although a 24 bp fragment would also result. The 1499 bp p-hydroxyphenylpyruvate dioxygenase band and the 2863 bp T7BlueR band were cut out of the gel and the associated DNA purified from the agarose using a QIAquick Gel Extraction Kit (Qiagen, Chatsworth, Calif.) according to the manufacturer's instructions. The purified DNA samples were precipitated by the addition of sodium acetate (pH 5.2) to 0.3 M, 10 μg tRNA (added as carrier), two volumes of −20° C. ethanol and incubation at −20° C overnight. Nucleic acid pellets were collected by centrifugation, washed with 70% ethanol and air dried. Both pellets were solublized in 10 μL of TRIS/EDTA (TE) buffer, pH 8 (Maniatis), and then 1 μL of each sample loaded onto a 1% agarose, TAE gel in separate wells next to a well containing 4 μL of Mass Ladder (Gibco BRL). All samples were adjusted to 10 μL with water before loading. DNA was quantified by comparing band intensities of each sample with Mass Ladder band intensities following ethidium bromide staining and UV illumination.

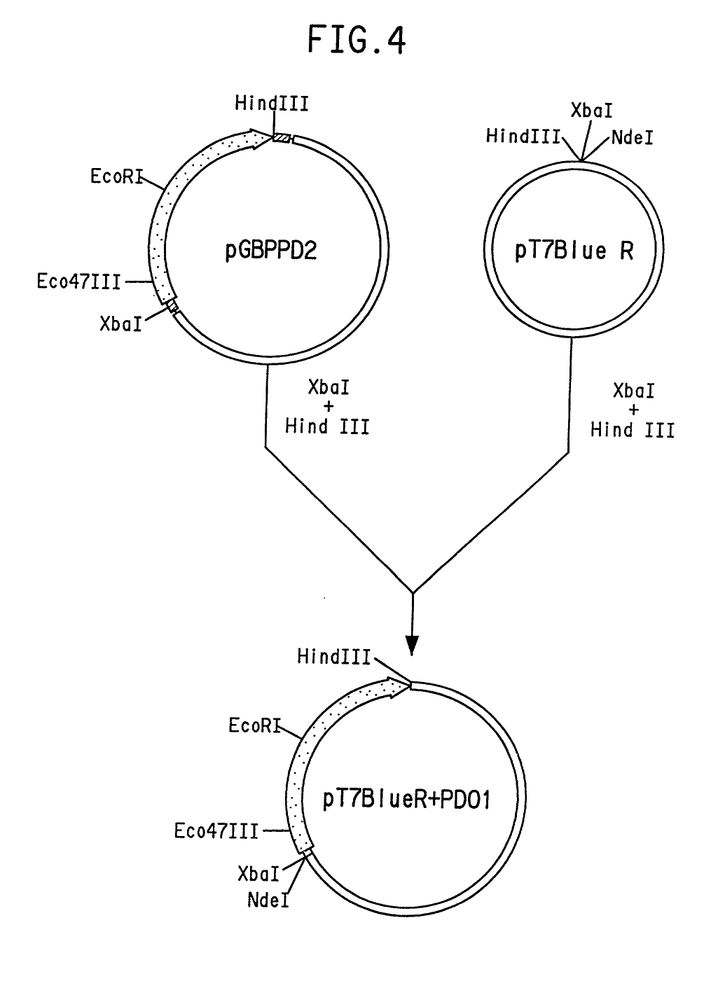

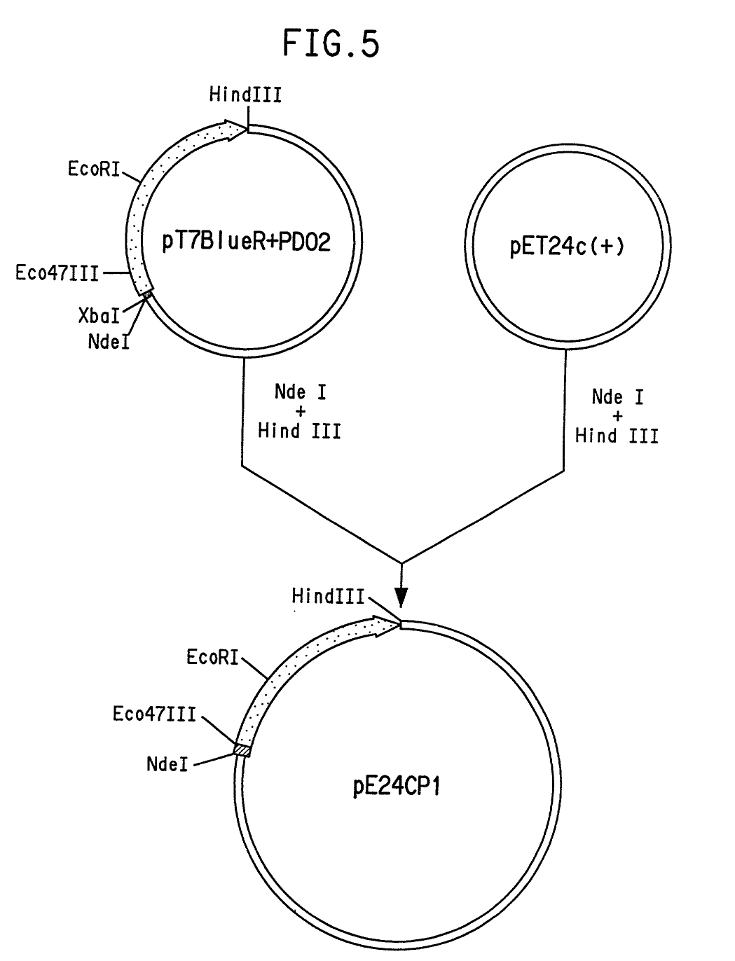

Approximately 300 ng of p-hydroxyphenylpyruvate dioxygenase insert was mixed with 300 ng of double digested pT7BlueR vector in a total volume of 7 μL and then heated to 45° C. for 5 min followed by cooling on ice. T4 DNA ligase buffer (Gibco BRL) and 1 unit of T4 DNA ligase (Gibco BRL) were added to the cooled DNA for a total volume of 10 μL. The ligation mix was incubated at room temperature for 4 h and then transformed into MAX Efficiency DH5α Competent Cells (Gibco BRL) of E. coli according to standard procedures (Maniatis). Transformed bacteria were spread onto LB agar plates supplemented with 100 μg/mL carbenicillin and incubated overnight at 37° C. Seventeen bacterial colonies were selected for subsequent analysis. A portion of each colony was inoculated into a separate 17×100 mm polypropylene culture tube (Falcon, Lincoln Park, N.J.) containing 2 mL of liquid LB media and 200 μg/mL carbenicillin. Liquid bacteria cultures were incubated overnight at 37° C. with shaking (250 rpm). Plasmid DNA was then isolated using a QIAprep Spin Plasmid Miniprep Kit (Qiagen) according to the manufacturer's instructions. A portion (5 μL out of 50 μL total) of each plasmid preparation was digested with 10 units each of Hind III and EcoRV (Gibco BRL) in a total volume of 15 μL with React 2 buffer (Gibco BRL) for one h. (Note: The EcoRV site in the pBluescript polylinker was destroyed during the preparation of pGBPPD2 so only the EcoRV site in the pT7BlueR polylinker would be accessible to the restriction nuclease). Samples were separated electrophoretically in 1% agarose and tris/borate/EDTA (TBE) buffer (Maniatis). Bands were visualized with ethidium bromide staining; 7 out of 17 samples which contained 2 bands (2837 and 1525 bp) contained the p-hydroxyphenylpyruvate dioxygenase insert and were designated pT7BlueR+PDO1 (see FIG. 4).

In order to remove the putative chloroplast transit sequence, the remaining 45 μL of each prep of pT7BlueR+PDO1 were combined into a single sample and the DNA content determined spectrophotometrically at A₂₆₀ (Maniatis). A portion (5 μg) of pT7BlueR+PDO 1 was digested with 16 units of Eco47 III (MBI Fermentas) in a total volume of 100 μL containing buffer 0 (MBI Fermentas) at 37° C. for 2 h. The digested plasmid DNA was then precipitated with sodium acetate and ethanol as above and the resulting dried nucleic acid pellet was dissolved in 60 μL of React 2 (Gibco BRL) containing 20 units of Nde I (Gibco BRL) and incubated 2 h at 37° C. The double digested sample was then loaded onto a 1% agarose gel in TAE and the large 4166 bp Nde I-Eco47III fragment separated from the 196 bp fragment electrophoretically. The large fragment was cut out of the gel, purified from agarose and precipitated as above.

An oligonucleotide mix was prepared consisting of 100 pmoles each of oligos CAM32 and CAM33 (SEQ ID NOS: 4 and 5, respectively) in a combined volume of 9.9 μL. The two oligos complement each other to form a 3′ blunt end corresponding to the 5′ half of an Eco47 III restriction site and also form a 5′ staggered end which corresponds to the 3′ half of an Nde I restriction site.

| | |
|---|---|
| CAM 32: | (SEQ ID NO:4) |

5′-TATGTCCAAGTTCGTAAGAAAGAATCCAAAGTCTGATAAATTCAAGGT

TAAGC-3′

| | |
|---|---|
| CAM 33: | (SEQ ID NO:5) |

5′-GCTTAACCTTGAATTTATCAGACTTTGGATTCTTCTTACGAACTTGGA

CA-3′

The oligo mix was heated to 90° C. for 1.5 min and then allowed to cool to room temperature over 20 min. The dried nucleic acid pellet resulting from purification of the 4166 bp Nde I-Eco47 III fragment was solublized in 7 μL of the cooled oligo mix and subsequently heated to 45° C. for 5 min followed by cooling on ice. Ligation of the oligos with the Nde I-Eco47 III fragment followed by transformation into DH5α was performed as above. Transformed bacterial cells were spread onto LB/carbenicillin plates and incubated at 37° C. overnight. Seventeen colonies were selected and processed to isolate plasmid DNA as above. A portion (5 out of 50 μL) of each plasmid was double digested with 10 units each of Nde I and Hind III and the fragments separated electrophoretically on a 1% agarose gel in TBE. A two band pattern corresponding to insert (1373 or 1518 bp) and vector (2844 bp) was detected. An additional double digest with 10 units each of Xba I and Hind III was performed on another 5 μL aliquot of plasmids. When digested with Nde I and Hind III, none of the plasmids which contained the smaller insert size contained a Xba I site. The Xba I site would be eliminated if the two oligos replaced the 196 bp fragment originally present in pT7Blue+PDO 1. The 7 plasmid samples with the modified p-hydroxyphenylpyruvate dioxygenase insert were combined and designated pT7BlueR+PDO2.

The pT7BlueR+PDO2 plasmid DNA was quantified spectrophotometrically (above) and then 5 μg was digested with 20 units each of Hind III and Nde I in 62 μL of React 2 for 2 h at 37° C. The digested sample was subsequently loaded onto a 1% agarose gel in TAE and separated electrophoretically. The 1373 bp fragment was isolated and precipitated as above. The plasmid pET24c(+) (5 μg) was double digested with 20 units each of both Nde I and Hind III in React 2 at 37° C. for 2 h and the 5245 bp fragment then gel purified on a 1% agarose gel in TAE and subsequently separated from agarose and precipitated as above. The dried pET24c(+) pellet was solublized in 10 μL TE and then 8 μL was adjusted to a 20 μL total volume with water, dephosphorylation buffer (Gibco BRL) and 1 unit of calf intestinal alkaline phosphatase (Gibco BRL). The sample was incubated at 37° C. for 30 min and then gel purified, separated from agarose, and precipitated as above. The dried, dephosphorylated, pET24c(+) vector pellet and modified p-hydroxyphenylpyruvate dioxygenase insert pellet were each solublized in 10 μL TE and then 1 μL of each was run on a 1% agarose TBE gel with 4 μL of mass ladder to quantify DNA as above. One hundred nanograms of modified p-hydroxyphenylpyruvate dioxygenase insert was mixed with 120 ng of dephosphorylated pET24c(+) vector in a total of 7 μL volume. The mix was heated to 45° C. for 5 min and then cooled on ice. The mix was then supplemented with T4 DNA ligase buffer and 1 unit of T4 DNA ligase in a total volume of 10 μL and the mix allowed to incubate at room temperature for 4 h. The ligation mix was subsequently transformed into DH5α, spread on LB agar supplemented with 30 μg/mL kanarnycin, and incubated overnight at 37° C. Plasmid preparations were performed on 11 colonies as above. Plasmids were double digested with Nde I and Hind III and fragments separated electrophoretically. All plasmids had the expected 1373 bp and 5245 bp fragments. One bacteria colony was selected and used to inoculate 100 mL of liquid LB supplemented with 30 μg/mL kanamycin which was subsequently incubated at 37° C. overnight with shaking. Plasmid DNA was isolated from the resulting bacteria culture using a Qiagen Plasmid Midi Kit according to the manufacturer's instructions. A portion of the plasmid DNA (pE24CP1) was sequenced with the Sequenase Version 2.0 DNA Sequencing Kit (United States Biochemical, Cleveland, Ohio) using a biotinylated sequencing primer to the T7 promoter (United State Biochemical) according to the manufacturer's instructions for non-radioactive manual sequencing. DNA was transferred from the sequencing gel to Hybond-N+ nylon transfer membrane (Amersham, Arlington Heights, Ill.) by capillary action. Transfer and all subsequent steps in chemiluminescent detection of DNA fragments were performed with a SEQ-Light Chemiluminescent Sequencing System kit (Tropix, Bedford, Mass.) according to the manufacturer's instructions. DNA sequencing verified that the plasmid contained the expected 5′ sequence for the modified p-hydroxyphenylpyruvate dioxygenase insert where nucleotides 1-95 (FIG. 2) were replaced with an ATG transcriptional start site. This is equivalent to amino acids 2-29 (FIG. 3) being eliminated from the N-terminus of the Arabidopsis p-hydroxyphenylpyruvate dioxygenase amino acid sequence.

The plasmid pE24CP1 was transformed into competent cells of BL21(DE3) E. coli (Novagen), as above. Transformed cells were spread on LB/kanamycin plates and incubated overnight at 37° C. Seven colonies were selected for plasmid preparations as above and plasmid DNA was double digested with Nde I and Hind III to verify that all plasmids had the expected electrophoretic banding pattern. One colony was selected and streaked for isolation on LB/kanamycin plates. A well isolated colony was used to inoculate liquid LB supplemented with 30 μg/mL kanamycin and the culture was incubated at 37° C. with shaking (250 rpm) until it reached an A₆₀₀ of 0.6 absorbance units. An 8% glycerol freezer stock was prepared according to the Novagen protocol and stored at −80° C. All subsequent expression studies were done with freshly grown bacterial cells that were isolated from LB/kanamycin plates streaked from the glycerol freezer stock.

BL21(DE3) E. coli cells containing either pE24CP1 or pET 24c(+) (negative control) were streaked out onto LB/kanamycin plates from a glycerol freezer stock (above) and incubated overnight at 37° C. One isolated colony was selected for inoculation of 2 mL of LB containing 30 μg/mL kanamycin in a 17×100 mm Falcon tube, and the culture was incubated at 37° C. with shaking (250 rpm) overnight. The overnight cultures were then used to inoculate 100 mL of fresh LB containing 30 μg/mL kanamycin. The new cultures were incubated at 37° C. with shaking until the A₆₀₀ reached between 0.4 and 0.6 absorbance units. One half of the pE24CP 1 and pET24c(+) cultures were placed in new culture flasks and IPTG (isopropylthio-β-D-galactoside; Gibco BRL) was added to the new flasks to give a final concentration of 1 mM. The flasks were incubated an additional 3 h at 37° C. with shaking, and then the cells were harvested.

The harvested cells were centrifuged and the resulting cell pellet extracted by sonication (3×10 sec bursts) in 2 mL extraction buffer (50 mM (20 mM in the first experiment; Table 2) potassium phosphate buffer, pH 7.2, containing 0.14 M KCl, 0.32 mM reduced glutathione, 1% polyvinylpolypyrrolidone, and 0.1% Triton X 100 (0.01% lysozyme was included in the first experiment only)). The lysate represents the crude extracted enzyme after centrifugation at 17000 g for 10 min. In the first experiment (Table 2) a 20 to 60% ammonium sulfate precipitated enzyme fraction was also assayed. Solid ammonium sulfate was slowly added with stirring to 2 mL of the lysate to bring the concentration to 20% (w/v). After incubation on ice for approximately 15 min, the solution was centrifuged at 17000 g for 10 min. The supernatant liquid was harvested and solid ammonium sulfate was added to increase the concentration to 60% (w/v). After centrifugation, the resulting pellet was resuspended in 1 mL of the extraction buffer.

A portion of the insoluble protein resulting from expression of Arabidopsis p-hydroxyphenylpyruvate dioxygenase in bacteria was utilized for N-terminal sequence analysis. The protein (approximately 180 μg) was suspended in 60 μL of extraction buffer and then diluted with 5 volumes of sample buffer (62.5 mM Tris, pH 6.8, 6 M urea, 160 mM dithiothreitol, 0.01% bromophenol blue) followed by intermittent vortexing for one hour at room temperature. A 1.5 mm thick, 12% polyacrylamide resolving gel was prepared for a Mini-Protein II dual slab cell (Bio-Rad, Hercules, Calif.) using the manufacturer's instructions. The polyacrylamide was allowed to polymerize for 3 h and then a stacking gel was prepared using a preparative comb. The running buffer was prepared according to the manufacturer's instructions with the addition of 0.1 mM sodium thioglycolate. The solublized protein sample was electrophoretically separated using the manufacturer's instructions. When the bromophenol blue dye front reached the bottom of the gel, the gel was removed and equilibrated for 5 min in blotting buffer (10 mM CAPS, pH 11, 10% methanol, balance water). The gel was then placed in a Mini Trans-Blot Electrophoretic Transfer Cell (Bio-Rad), according to the manufacturer's instructions, with a ProBlott PVDF membrane (Applied Biosystems, Foster City, Calif.) treated according to the manufacturer's instruction. Electroblotting was done in the presence of blotting buffer at 50 volts for 45 min in an ice bath. The membrane was then rinsed in water and stained with Coomassie Blue as described in the ProBlott protocol. The major protein band was excised from the membrane and subjected to N-terminal amino acid sequencing on a Beckman (Fullerton, Calif.) LF3000 protein sequencer. The first 11 cycles identified S-K-F-V-R-K-N-P-K-S-D (see SEQ ID NO: 3, amino-acids 30-40), respectively. This is the expected N-terminus of the modified Arabidopsis p-hydroxyphenylpyruvate dioxygenase minus the initial methionine (amino acids 30-40, FIG. 3).

### EXAMPLE 3

### p-Hydroxyphenylpyruvate Dioxygenase Enzymatic Activity of the Plant Protein Expressed in E. Coli

Cell cultures with different plasmid constructs were extracted as described above and assayed by measuring the formation of ¹⁴Co₂ from [1-¹⁴C]-p-hydroxyphenylpyruvate or ¹⁴CO₂ and ¹⁴C-homogentisate from [U-¹⁴C]-p-hydroxyphenylpyruvate (Lindblad, B., (1971) Clin. Chim. Acta 34:113-121; and Lindstedt, S. and Odelhog, B., (1987) Methods in Enzymology 142:143-148). The labeled substrate was prepared from [1-¹⁴C]-L-tyrosine (55 mCi/mmol; American Radiolabeled Chemicals, Inc., St. Louis, Mo.) or [U-¹⁴C]-L-tyrosine (498 mCi/mmol; DuPont NEN, Boston, Mass.). A 50-100 μL aliquot (5-10 μCi) of the of the labeled tyrosine stock solution was transferred to a 4 mL glass vial and blown to dryness in a stream of nitrogen at 45° C. To the vial was added 175 μL of 0.1 M phosphate buffer, pH 6.5, 5 μL catalase (28,700 units of C-100, Sigma Chemical Co., St. Louis, Mo.), and 20 μL L-amino acid oxidase (Sigma A-9253, 6.5 units/mL). The vial was then placed on a shaker water bath set at 30° C., 60 cycles/min, for 0.5 to 1 h. The reaction mix was then passed through a small column containing 400 μL Dowex AG 50W X8 cation exchange resin. The column was then washed with 1.5 mL of water and the eluant containing the labeled p-hydroxyphenylpyruvate was collected. The labeled substrate was either used immediately or stored at −80° C. and used within a week after preparation.

The assay was performed in 14 mL culture tubes capped with serum stoppers through which a polypropylene well containing 200 μL of 1 N KOH was suspended. The reaction mixture contained 5,740 units of catalase, 100 μL of a freshly prepared 1:1 (v:v) mixture of 150 mM reduced glutathione and 3 mM dichlorophenolindophenol, 5 mM ascorbate, 0.1 mM ferrous sulfate (the ascorbate and ferrous sulfate were not present in the buffer used in the first experiment; Table 2), 50 μM unlabeled p-hydroxyphenylpyruvate, 1-25 μL of the enzyme extract, and 50 mM potassium phosphate buffer in a final volume of 980 μL. Unlabeled substrate was made fresh daily in 50 mM potassium phosphate buffer and allowed to equilibrate for at least 2 h at room temperature to insure that greater than 95% was in the keto form. The tubes were incubated for 10 min at 30° C. in a shaking water bath prior to adding 20 μL (0.04 μCi) of ¹⁴C-p-hydroxyphenylpyruvate. The reaction was terminated after 60 min by injecting 500 μl of 1 N sulfuric acid through the serum stopper. The vials were left on the shaker for another 30 min to insure complete capture of the released ¹⁴CO₂. The serum caps were then removed and the wells cut and dropped into 8 mL scintillation vials. Six mL of Formula-989 scintillation fluid (Packard Insturments, Meriden, Conn.) was added to the vials and the ¹⁴C radioactivity was determined by scintillation counting. Table 2 summarizes the results of this experiment.

TABLE 2
p-Hydroxyphenylpyruvate Dioxygenase Activity of Extracts from
E. coli Containing Different Plasmid Constructs

| | Inducer | Lysate | | Ammonium Sulfate Precipitate | |
|---|---|---|---|---|---|
| Plasmid | (1 mM IPTG) | dpm * /mg | nmol/min × mg | dpm * /mg | nmol/min × mg |
| pET24c(+) | − | 12,318 | 0.09 | 0 | 0.00 |
| pET24c(+) | + | 35,115 | 0.25 | 3,393 | 0.03 |
| pE24CP1 | − | 24,607 | 0.17 | 126,761 | 0.89 |
| pE24CP1 | + | 243,801 | 1.71 | 1,371,823 | 9.64 |

* ¹⁴C : ¹²C = 1 : 50; sp. act. of ¹⁴C-p-hydroxyphenylpyruvate = 55 mCi/mmol

The results show there was little or no p-hydroxyphenylpyruvate dioxygenase activity in any of the cell cultures that did not have the plasmid containing the nucleic acid fragment encoding p-hydroxyphenylpyruvate dioxygenase (pET24c(+)) and the inducer of gene expression (IPTG). The gene and inducer together resulted in a marked increase in activity.

In the experiment with [U- 14C] p-hydroxyphenylpyruvate (“HPPA”), where both ¹⁴CO₂ and ¹⁴C-homogentisic acid were measured, the reaction was initiated by adding 50 μL of labeled substrate (0.3 μCi) and was terminated with 100 μL of 10% phosphoric acid. The ¹⁴CO₂ released was determined by scintillation counting, while the level of homogentisic acid was determined by HPLC on a Zorbax RX-C8 column (4.6×250 mm) with an in-line radioactivity detector. Aliquots of 1.7 to 15 μL were taken from the reaction mix after centrifugation and diluted into the column equilibration buffer prior to injection. Separation was performed at ambient temperature with a flow rate of 1.0 mL/min and the following gradient with solvent A and B being water and methanol, each with 1% phosphoric acid: 0-2 min, isocratic at 95% A and 5% B; 2-17 min, linear gradient from 95 to 75% A and 5 to 25% B; 17-19 min linear gradient from 75 to 5% A and 25 to 95% B; 19-22 min, isocratic at 5% A and 95% B; 22-24 min, linear gradient from 5% to 95% A and 95 to 5% B. In this system homogentisate eluted at 10.8 min. The results from this experiment are shown in Table 3.

TABLE 3
p-Hydroxyphenylpyruvate Dioxygenase Activity of Cell Extracts
Determined by CO₂Release and Homogentisic Acid Synthesis
from [U-¹⁴C] p-Hydroxyphenylpyruvate

| | |
|---|---|
| Inducer | nmol/min × mg* |

| | | | |
|---|---|---|---|
| Plasmid | (1 mM IPTG) | ¹⁴CO₂ | Homogentisic acid |

| | | | |
|---|---|---|---|
| pET24c(+) | − | 0.00 | 0.00 |
| pET24c(+) | + | 0.19 | 0.00 |
| pE24CP1 | − | 4.68 | 4.76 |
| pE24CP1 | + | 29.12 | 29.82 |

*¹⁴C:¹²C = 1:87.7; sp. act. of ¹⁴C[U]-p-HPPA = 498 mCi/mmol

There was a tight correlation between the results from the assays of the two products of the reaction. The results confirmed there was no significant p-hydroxyphenylpyruvate dioxygenase activity in either cell culture that did not contain the nucleic acid fragment encoding p-hydroxyphenylpyruvate dioxygenase. There was measureable enzyme activity in the absence of the inducer, but when the inducer was added the activity increased greater than six-fold over uninduced cultures. These results and those of Table 2 clearly show that the nucleic acid fragment isolated and overexpressed in E. coli cells encodes a protein that catalyzes the conversion of p-hydroxyphenylpyruvate to homogentisate with the release of CO₂.

The overexpressed protein was also assayed spectrophotometrically at ambient temperature using the enol borate-tautomerase assay (Lin, E. C. C. et al., (1958) J. Biol. Chem. 233:668-673). The assay buffer contained 0.4 M borate (adjusted to pH 7.2 with 0.2 M sodium borate), 4 mM ascorbate, 2.5 mM EDTA, 40 μM p-hydroxyphenylpyruvate, and 0.5 units of tautomerase (Sigma T-6004) per 10 mL buffer. The reaction mix was used when the tautomerization of the substrate was complete (when absorbance at 308 nm had stabilized). The assay was initiated by adding 40 μL of the cell extracts to 960 μL of the assay buffer, and the reaction was followed by measuring the decrease in absorbance at 308 nm.

Table 4 summarizes the results with extracts of the same four cell cultures described in Table 3.

TABLE 4
Spectrophotometric Assay of p-Hydroxyphenylpyruvate
Dioxygenase Activity of Cell Extracts

| | Inducer | |
|---|---|---|
| Plasmid | (1 mM IPTG) | nmol p-HP lost/min × mg* |
| pET24c(+) | − | 1.58 |
| pET24c(+) | + | 2.73 |
| pE24CP1 | − | 4.91 |
| pE24CP1 | + | 22.32 |

*Loss of p-hydroxyphenylpyruvate based on a molar extinction coefficient for the equilibrium mixture of 9850 as reported by Lin et al. ((1958) J. Biol. Chem. 233: 668-673).

### EXAMPLE 4

### Inhibition of p-Hydroxvphenylpyruvate Dioxygenase by Commercial Herbicides

The enzymatic activity of the overexpressed protein is inhibited by two herbicides known to inhibit plant p-hydroxyphenylpyruvate dioxygenase: Sulcotrione (2-(2-chloro-4-methanesulfonylbenzoyl)-1,3-cyclohexanedione); and Isoxaflutole (5-cyclopropylisoxazol-4-yl 2-mesyl-4-trifluoromethylphenyl ketone). These two compounds were tested against the overexpressed protein using both the ¹⁴CO₂ and the continuous spectrophotometric enol borate-tautomerase assays. Both compounds were added to the assay buffers in 10 μL of acetone or dimethyl sulfoxide. The I₅₀ values (concentration inhibiting the enzyme 50%) were calculated based on the percent inhibition observed over several concentrations of the inhibitor. The results of the assays are shown in Table 5.

TABLE 5
I₅₀ Values of Inhibitors of Plant p-Hydroxyphenylpyruvate Dioxygenase

I₅₀ value (nM) derived from

| Compound | ¹⁴CO₂ assay | spectrophotometric assay |
|---|---|---|
| sulcotrione | 43 | 44 |
| isoxaflutole | 409 | 1042 |

These results clearly show that the p-hydroxyphenylpyruvate dioxygenase activity of the overexpressed protein is inhibited by commercial herbicides that have inhibition of this enzyme as their mode of action. Moreover, the continuous spectrophotometric assay gave similar ISO values to those obtained with the ¹⁴CO₂ assay. The spectrophotometric assay can be adapted to a high capacity screen for inhibitors of p-hydroxyphenylpyruvate dioxygenase by adapting it to a microtiter plate assay combined with a plate reader that would read at or near 308 nm. Furthermore, any colorimetric or fluorescent assay for homogentisate or p-hydroxyphenylpyruvate would also be able to be readily adapted into a high capacity screen for inhibitors of this enzyme. The isolated overexpressed enzyme has sufficient activity to be used directly in a spectrophotometric assay or it can be further purified for enhanced assay sensitivity.

### EXAMPLE 5

### Re-construction of the Full-length p-Hydroxyphenylpyruvate Dioxygenase Gene for Production of Active, Stable Enzyme in Bacteria

The plasmid pT7BlueR+PDO2, described in Example 2 and containing the full-length p-hydroxyphenylpyruvate dioxygenase gene, proved to have incorrect sequence at the EcoRI site. This was re-sequenced so that an oligonucleotide could be designed to replace the EcoRI site with an NdeI site using conventional loop-out mutagenesis. The oligonucleotide was designed so that this procedure also introduced an ATG initiation codon at the 5′-end of the p-hydroxyphenyl-pyruvate dioxygenase gene followed by the full-length p-hydroxyphenylpyruvate dioxygenase sequence. After mutagenesis, the clone was amplified in E. coli and the plasmid was purified. The resulting full-length gene, “PDO-B”, was then digested with the enzymes using NdeI and NheI, and the ˜820 bp fragment used to replace the NdeI- NheI segment of the truncated p-hydroxyphenylpyruvate dioxygenase gene, “PDO-A,” in pE24CP1 (Example 1). The resulting plasmid, pE24PDO-B can be expressed in bacteria to produce the full-length Arabidopsis p-hydroxyphenylpyruvate dioxygenase enzyme as determined by enzyme activity and N-terminal sequence analysis.

### EXAMPLE 6

### Enhanced Stability of Full Length Construct Over the Truncated Construct

Two different constructs for Arabidopsis thaliana p-hydroxyphenyl-pyruvate dioxygenase, one containing the full-length sequence, PDO-B as described in Example 5 and produced from plasmid pE24PDO-B, and one containing the truncated sequence lacking the putative chloroplast leader sequence, PDO-A as produced from plasmid pE24CP1, were both purified to the same extent using a Pharmacia phenyl Sepharose hydrophobic interaction column followed by gel filtration chromatography on Pharmacia Sephacryl 300. The two proteins were diluted to 1 mg/mL in 20 mM bis tris-propane buffer, pH 7.2 containing 5 mM ascorbate, 1 mM reduced glutathione and 0.1mM ferrous ammonium sulfate and stored in a refrigerator at 4 ° C. for up to 10 days. Aliquots were removed at various times and assayed for activity using the tautomerase coupled spectrophotometric assay. Under these conditions the half-life for the activity of the full length enzyme was 4 days, whereas the truncated enzyme preparation had a half-life of 9 to 10 hours. In addition, the activity of the full length enzyme could be restored by incubation with iron and reducing agent, reduced glutathione or ascorbate, or by dialysis against buffer containing iron and reducing agent. In contrast, the activity of the truncated enzyme could not be restored by incubation with or dialysis against buffer containing iron and reducing agent. The full-length enzyme was also more stable in the spectrophotometric assay showing a 2 to 3 times longer useful linear region than the truncated enzyme. Both enzyme preparations showed similar I₅₀ values with the herbicidally active inhibitors.

These results clearly show that the full-length PDO-B construct has decided advantages over the truncated enzyme due to the enhanced stability under storage conditions, in the spectrophotometric assay and in the reversible reconstitution of activity in the presence of iron and reducing agent. While both enzyme constructs can be used for screening of inhibitors, the PDO-B enzyme is preferred for this application and is far superior for mechanistic and structural studies.

### EXAMPLE 7

### Cloning of the Maize p-Hydroxyphenylpyruvate Dioxygenase Gene

Approximately 600,000 plaques of a Stratagene maize Uni-Zap cDNA library (from young plants) were screened by filter hybridization under moderate stringency using a heterologous probe. The probe was prepared by PCR and was a 916 bp fragment of DNA having the sequence defined by the region extending from position 263 to 1178 of SEQ ID NO: 14. Twenty-four positive phage clones were identified in the primary screen, and eleven phage clones were recovered from a secondary screen. Seven positive clones were submitted for sequencing, and four showed significant conservation sequence at the amino acid level when compared with the Arabidopsis thaliana p-hydroxyphenylpyruvate dioxygenase protein. The longest of the four contained an insert of 988 bp and showed 70% identity and 78% similarity with the Arabidopsis protein, but was lacking approximately 550 bp corresponding to the amino terminal end of the protein.

Attempts to obtain a full-length cDNA of the maize p-hydroxyphenyl-pyruvate dioxygenase gene were unsuccessful, possibly because the secondary structure of the RNA inhibited efficient reverse transcription of this transcript. Two additional cDNA libraries were screened and clones long enough to contain a full-length cDNA were sequenced. All of these clones were shown to be chimeras. Therefore a genomic library was screened to obtain the 5′ one-third of the gene. Approximately 1 million clones from a Clontech Zea mays (var. B73) library in the phage vector EMBL3 (whole seedlings, 2 leaf stage) were screened using a 415 bp EcoRI-BssHII fragment containing the 5′ end of the truncated corn p-hydroxyphenylpyruvate dioxygenase cDNA (clone H1011 C). Eight positive primary phage clones were plated and screened, and four secondary clones were picked. DNA was prepared from each using the Qiagen Lambda midi-kit. Restriction digests with SalI or EcoRI indicated that two clones were the same. DNA samples from the remaining 3 clones (11.1.3, 13.1.1, and 21.2.1) were digested with SalI, EcoRI, or SalI and EcoRI, prepared for Southern analysis, and probed with the full length Arabidopsis p-hydroxyphenylpyruvate dioxygenase gene. Two of the clones (11.1.3 and 13.1.1) showed sequence conservation, and these homologous fragments were subcloned and sequenced. Both clones appeared to contain the full-length gene and each contained one intron near the 3′ end of the gene. However, there were differences between the sequences of the two clones indicating that they may be two different genes or one may be a pseudogene. The sequence of clone 11.1.13 matched the cDNA sequence, and this clone was used to construct a full length p-hydroxyphenylpyruvate dioxygenase coding region.

The gene was contained on two adjacent fragments, a 3.5 kb EcoRI - SalI fragment and a 2 kb SalI fragment. Both were subcloned into pBluescript SKII+ resulting in the plasmids pES1113 and pSal1113. pES1113 was digested with SpeI to release approximately 2.7 kb of upstream sequence and then religated, resulting in a plasmid with an insert of 747 base pairs (PSPE1). pSPE1 was digested with SalI to linearize the plasmid and ligated with the 2 kb SalI fragment from pSal 1113, which had been released by digestion with SalI and gel purified. Orientation was confirmed by digestion with SpeI and Bpu 1102I and the correct plasmid was named p1113. In order to remove the intron contained in the 3′ end of the genomic clone, the plasmid was digested with Bpu 1102I and XhoI and the 3.9 kb fragment containing the vector and 5′ part of the gene was gel purified. The corresponding 882 bp Bpu 1102I -XhoI fragment from pH1011c (cDNA)was gel purified and ligated with this 3.9 kb fragment resulting in the clone pMPDO (ATCC 209120), which contains a 1782 bp insert. There are 260 base pairs upstream of the putative ATG and 189 base pairs downstream of the stop codon. The full-length sequence was confirmed by sequencing across the insert. The nucleic acid sequence and the deduced protein sequence for corn p-hydroxyphenylpyruvate dioxygenase are presented in SEQ ID NOS: 10 and 11, respectively. The sequences for p-hydroxyphenylpyruvate dioxygenases obtained from corn and Arabidopsis were compared using the “Gap” program of GCG (Program Manual for the Wisconsin Package, Version 9.0-OpenVMS, December 1996, Genetics Computer Group, 575 Science Drive, Madison, Wis., USA 53711). The results of these comparisons indicated that these functions are approximately 67% identical at the nucleotide level, and they possess 69% similarity and 62% identity at the amino acid level. The predicted amino acid sequence of corn p-hydroxyphenylpyruvate dioxygenase is compared with that from Arabidopsis and other eukaryotes in FIG. 3.

### EXAMPLE 8

### Composition of a cDNA Library, Isolation and Sequencing of cDNA Clones

A cDNA library representing mRNAs from developing seeds of Vernonia galamenensis that had just begun production of vernolic acid was prepared. The library was prepared in a Uni-ZAP™ XR vector according to the manufacturer's protocol (Stratagene Cloning Systems, La Jolla, Calif.). Conversion of the Uni-ZAP™ XR library into a plasmid library was accomplished according to the protocol provided by Stratagene. Upon conversion, cDNA inserts were contained in the plasmid vector pBluescript. cDNA inserts from randomly picked bacterial colonies containing recombinant pBluescript plasmids were amplified via polymerase chain reaction using primers specific for vector sequences flanking the inserted cDNA sequences. Amplified insert DNAs were sequenced in dye-primer sequencing reactions to generate partial cDNA sequences (expressed sequence tags or “ESTs”; see Adams, M. D. et al., (1991) Science 252:1651). The resulting ESTs were analyzed using a Perkin Elmer Model 377 fluorescent sequencer.

### EXAMPLE 9

### Identification and Characterization of cDNA Clones

ESTs encoding Vernonia galamenensis enzymes were identified by conducting BLAST (Basic Local Alignment Search Tool; Altschul, S. F. et al., (1993) J. Mol. Biol. 215:403-410; see also www.ncbi.nlm.nih.gov/BLAST/) searches for similarity to sequences contained in the BLAST “nr” database (comprising all non-redundant GenBank CDS translations, sequences derived from the 3-dimensional structure Brookhaven Protein Data Bank, the last major release of the SWISS-PROT protein sequence database, EMBL, and DDBJ databases). The cDNA sequences obtained in Example 9 were analyzed for similarity to all publicly available DNA sequences contained in the “nr” database using the BLASTN algorithm provided by the National Center for Biotechnology Information (NCBI). The DNA sequences were translated in all reading frames and compared for similarity to all publicly available protein sequences contained in the “nr” database using the BLASTX algorithm (Gish, W. and States, D. J. (1993) Nature Genetics 3:266-272) provided by the NCBI. For convenience, the P-value (probability) of observing a match of a cDNA sequence to a sequence contained in the searched databases merely by chance as calculated by BLAST are reported herein as “pLog” values, which represent the negative of the logarithm of the reported P-value. Accordingly, the greater the pLog value, the greater the likelihood that the cDNA sequence and the BLAST “hit” represent homologous proteins.

The BLASTX search using clone vs1.pk0015.b2 revealed similarity of the protein encoded by the cDNA to a number of p-hydroxyphenylpyruvate dioxygenases from sources other that plants. The three most similar p-hydroxy-phenylpyruvate dioxygenase proteins were a streptomycete p-hydroxyphenyl-pyruvate dioxygenase (GenBank Accession No. U 11864; pLog=8.34), a rat p-hydroxyphenylpyruvate dioxygenase (GenBank Accession No. M 18405; pLog=7.66), and a human p-hydroxyphenylpyruvate dioxygenase (GenBank Accession No. U29895; pLog=7.60). SEQ ID NO: 16 shows the nucleotide sequence of a portion of the Vernonia galamenensis cDNA in clone vs1.pk0015.b2. Sequence alignments and BLAST scores and probabilities indicate that the instant nucleic acid fragment encodes a portion of Vernonia galamenensis p-hydroxyphenylpyruvate dioxygenase.

1. An isolated nucleic acid fragment encoding a plant p-hydroxy-phenylpyruvate dioxygenase enzyme, the fragment comprising a nucleotide sequence selected from the group consisting of
nucleotide sequences encoding a polypeptide comprising the amino acid sequences set forth in SEQ ID NO: 3, SEQ ID NO: 11, SEQ ID NO: 13, and SEQ ID NO: 15 and
modified nucleotide sequences essentially similar to the nucleotide sequences of SEQ ID NO: 2, SEQ ID NO: 10, SEQ ID NO: 12 and SEQ ID NO: 14 containing deletions, insertions, or substitutions in the sequence that do not affect the functional properties of the encoded protein.

2. An isolated nucleic acid fragment encoding a plant p-hydroxyphenyl-pyruvate dioxygenase enzyme, the fragment comprising a nucleotide sequence as set forth in SEQ ID NO: 14.

3. A chimeric gene comprising the nucleic acid fragment of claims 1 or 2 operably linked to at least one suitable regulatory sequence.

4. The chimeric gene of claim 3 wherein at least one suitable regulatory sequence directs gene expression in a microorganism.

5. The chimeric gene of claim 3 wherein the at least one suitable regulatory sequence directs gene expression in a plant.

6. A plasmid vector comprising the nucleic acid fragment of claims 1 or 2 operably linked to at least one suitable regulatory sequence.

7. A transformed host cell comprising a host cell and the plasmid vector of claim 6.

8. The transformed host cell of claim 7 wherein the host cell is derived from a plant or is a microorganism.

9. The transformed host cell of claim 8 wherein the microorganism is E coli.

10. A transformed plant tolerant to contact with at least one compound that inhibits the rate of the reaction of p-hydroxyphenylpyruvate dioxygenase enzyme in a non-transformed plant, the transformed plant comprising the chimeric gene of claim 3 and a host plant.

11. The transformed plant of claim 10 wherein the host plant is a cereal crop plant.

12. A method to identify a compound useful for its ability to inhibit the rate of the reaction of p-hydroxyphenylpyruvate dioxygenase enzyme comprising:
(a) transforming a host cell with the plasmid vector of claim 6;
(b) facilitating expression of the nucleic acid fragment encoding the plant p-hydroxyphenylpyruvate dioxygenase enzyme;
(c) contacting the expressed enzyme from step (b) with a test compound; and
(d) evaluating the capacity of the test compound to inhibit the rate of the reaction of p-hydroxyphenylpyruvate dioxygenase enzyme.

13. The method of claim 12 wherein evaluating the capacity of the test compound to inhibit the rate of the reaction of p-hydroxyphenylpyruvate dioxygenase enzyme is accomplished by measuring oxygen utilization, carbon dioxide release, homogentisate production, loss of p-hydroxyphenylpyruvate or maleylacetoacetate production.

14. The method of claim 12 wherein the transformed host cell is an E. coli that comprises a chimeric gene encoding a plant p-hydroxyphenylpyruvate dioxygenase enzyme.

15. A compound that inhibits the activity of a plant p-hydroxyphenylpyruvate dioxygenase enzyme, the compound identified by the method of claim 14.

16. A method for imparting tolerance to a plant to at least one compound that inhibits the rate of reaction of p-hydroxyphenylpyruvate dioxygenase enzyme comprising:
(a) transforming a host plant cell with a chimeric gene comprising a nucleic acid fragment encoding plant p-hydroxyphenylpyruvate dioxygenase, and
(b) expressing the chimeric gene in an amount effective to render the transformed plant substantially tolerant to the at least one compound that inhibits the rate of reaction of p-hydroxyphenylpyruvate dioxygenase.

17. A method for the microbial production of active plant p-hydroxyphenylpyruvate dioxygenase enzyme comprising:
(a) stably transforming a microorganism with the chimeric gene of claim 4 encoding the plant p-hydroxyphenylpyruvate dioxygenase;
(b) facilitating expression by the chimeric gene for a suitable period; and
(c) recovering active plant p-hydroxyphenylpyruvate dioxygenase enzyme.

18. A method to overexpress p-hydroxyphenylpyruvate dioxygenase enzyme in a plant comprising:
(a) stably transforming a host plant cell with a chimeric DNA molecule comprising at least one copy of a suitable regulatory sequence to drive expression of an associated coding sequence in a plant cell operably linked to at least one copy of a homologous or heterologous coding sequence encoding p-hydroxyphenylpyruvate dioxygenase; and
(b) growing the transformed host plant cell of step (a).

19. The method of claim 18 to overexpress p-hydroxyphenylpyruvate dioxygenase enzyme in a plant, wherein in step (a) the at least one copy of a homologous or heterologous coding sequence encoding p-hydroxyphenylpyruvate dioxygenase enzyme is selected from the group consisting of isolated nucleic acid fragments encoding a plant p-hydroxyphenylpyruvate dioxygenase enzyme, the fragment comprising a nucleotide sequence selected from the group consisting of:
(i) nucleotide sequences encoding a polypeptide comprising the amino acid sequences set forth in SEQ ID NO: 3, SEQ ID NO: 11, SEQ ID NO: 13, and SEQ ID NO: 15 and
(ii) modified nucleotide sequences essentially similar to the nucleotide sequences of SEQ ID NO: 2, SEQ ID NO: 10, SEQ ID NO: 12 and SEQ ID NO: 14 containing deletions, insertions, or substitutions in the sequence that do not affect the functional properties of the encoded protein.

20. An isolated nucleic acid fragment comprising a member selected from the group consisting of:
(a) an isolated nucleic acid fragment as set forth in SEQ ID NO: 16;
(b) an isolated nucleic acid fragment that is essentially similar to an isolated nucleic acid fragment as set forth in SEQ ID NO: 16; and
(c) an isolated nucleic acid fragment that is complementary to (a) or (b).
